(19) United States
(12) Patent Application Publication
(10) Pub. No.: US 2008/0108560 A1
(43) Pub. Date: May 8, 2008

(54) Heterologous G-Csf Fusion Proteins

(75) Inventors: John Michael Beals, Indianapolis, IN (US); Uma Kuchibhotla, Indianapolis, IN (US)

Correspondence Address: ELI LILLY & COMPANY, PATENT DIVISION, P.O. BOX 6288, INDIANAPOLIS, IN, 46206-6288, US

(73) Assignee: ELI LILLY AND COMPANY, Indianapolis, IN (US)

(21) Appl. No.: 10/506,455

(22) PCT Filed: Feb. 21, 2003
(86) PCT No.: PCT/US03/03120

§ 371 Date: Aug. 31, 2004

Related U.S. Application Data
(60) Provisional application No. 60/361,948, filed on Mar. 5, 2002.

(51) Int. Cl. A61K 38/00; A61P 43/00; C07H 21/04; C07K 16/00; C12N 15/00; C12N 5/04

(52) U.S. Cl. 514/12; 435/320.1; 435/358; 530/350; 536/23.1; 536/23.4

(57) ABSTRACT

The present invention encompasses heterologous fusion 5 proteins comprising a hyperglycsoylated G-CSF analog fused to proteins such as albumin and the Fc portion of animmunoglobulin which act to extend the in vivo half-life of the protein compared to native G-CSF. These fusion proteins are particularly suited for the treatment of conditions 10 treatable by stimulation of circulating neutrophils, such as after chemotherapy regimens or in chronic congenital neutropenia.

The present invention relates to heterologous fusion proteins, including analogs and derivatives thereof, fused to proteins that have the effect of extending the in vivo half-life of the proteins. These fusion proteins are significant in human medicine, particularly in the treatment of conditions treatable by stimulation of circulating neutrophils, such as after chemotherapy regimens or in chronic congenital neutropenia. More specifically, the invention relates to novel heterologous fusion proteins with granulocyte-colony stimulating factor activity.

Among all blood cell lineages, the modulation of neutrophil and platelet production has been of highest interest to clinical oncologists and hematologists. Myelosuppression is the single most severe complication of cancer chemotherapy, and a major cause of treatment delay during multiple-cycle or combination chemotherapy. It is also the major dose-limiting factor for most chemotherapeutic agents. Due to the short half-lives of neutrophils in peripheral blood, life-threatening falls in neutrophil levels are seen after a number of conventional anti-tumor chemotherapy regimens.

The most prominent regulator of granulopoiesis is granulocyte-colony stimulating factor (G-CSF). G-CSF induces proliferation and differentiation of hematopoietic progenitor cells resulting in increased numbers of circulating neutrophils. G-CSF also stimulates the release of mature neutrophils from bone marrow and activates their functional state. [Souza L. M., et al. (1986) Science 232:61-65]. Thus, therapeutic proteins with G-CSF activity have tremendous value in situations where there are reduced circulating levels of neutrophilic granuloctyes.

However, the usefulness of therapy using G-CSF peptides has been limited by their short plasma half-life. Thus, they must be administered intravenously or subcutaneously at fairly frequent intervals (once or twice a day) in order to maintain their neutrophil stimulating properties. In addition, this short half-life limits the performance of the drug to traditional drug delivery systems. It would clearly benefit the treatment of patients with abnormally low neutrophils, and reduce the discomfort and inconvenience associated with frequent injections to provide a pharmaceutical agent that could be administered less frequently and optionally by alternative routes of administration. Thus, a need exists to develop agents that stimulate the production of mature neutrophils and are more optimal in their duration of effect.

The present invention overcomes the problems associated with delivering a compound that has a short plasma half-life in two respects. First, G-CSF is hyperglycosylated. The carbohydrate content of G-CSF is altered by substituting amino acids that can act as substrates for glycosylating enzymes in mammalian cells. Most significantly, the present invention encompasses fusion of these hyperglycosylated G-CSF analogs to another protein with a long circulating half-life such as the Fc portion of an immunoglobulin or albumin.

Compounds of the present invention include heterologous fusion proteins comprising a hyperglycosylated G-CSF analog fused to a polypeptide selected from the group consisting of
a) human albumin;
b) human albumin analogs; and
c) fragments of human albumin.

### Compounds of the present invention also include heterologous fusion proteins comprising a hyperglycosylated G-CSF analog fused to a polypeptide selected from the group consisting of

a) human albumin;
b) human albumin analogs; and
c) fragments of human albumin,
   wherein the hyperglycosylated G-CSF analog is fused to the polypeptide via a peptide linker.

Additional compounds of the present invention include a heterologous fusion protein comprising a hyperglycosylated G-CSF analog fused to a polypeptide selected from the group consisting of
a) the Fc portion of an immunoglobulin;
b) an analog of the Fc portion of an immunoglobulin; and
c) fragments of the Fc portion of an immunoglobulin.
   The G-CSF analog may be fused to the polypeptide via a peptide linker. It is preferable that the peptide linker is selected from the group consisting of:
a) a glycine rich peptide;
b) a peptide having the sequence [Gly-Gly-Gly-Gly-Ser]ₙ where n is 1, 2, 3, 4, or 5; and
c) a peptide having the sequence [Gly-Gly-Gly-Gly-Ser)₃

The present invention further provides data showing that these G-CSF analogs are glycosylated in mammalian cells and retain their activity.

One aspect of the present invention includes heterologous fusion proteins, wherein the hyperglycosylated G-CSF analogs have the Formula (I) [SEQ ID NO:1]

```
(I)
1 5 10 15
Thr Pro Leu Gly Pro Ala Ser Ser Leu Pro Gln Ser Phe Leu Leu Lys
                        20 25 30
Xaa Leu Glu Gln Val Arg Lys Ile Gln Gly Asp Gly Ala Ala Leu Gln
                35 40 45
Glu Lys Leu Cys Xaa Xaa Xaa Lys Leu Cys His Pro Glu Glu Leu Val
        50 55 60
Leu Leu Gly His Ser Leu Gly Ile Xaa Xaa Xaa Xaa Xaa Xaa Xaa Xaa
65 70 75 80
Xaa Xaa Xaa Xaa Xaa Gln Leu Ala Gly Cys Leu Ser Gln Leu His Ser
                                85 90 95
Gly Leu Phe Leu Tyr Gln Gly Leu Leu Gln Ala Leu Xaa Xaa Xaa Ser
                        100 105 110
Xaa Glu Leu Gly Pro Thr Leu Asp Thr Leu Gln Leu Asp Val Ala Asp
                115 120 125
Phe Ala Thr Thr Ile Trp Gln Gln Met Glu Glu Leu Gly Met Ala Pro
        130 135 140
Ala Leu Gln Pro Xaa Xaa Xaa Ala Met Pro Ala Phe Xaa Xaa Xaa Phe
145 150 155 160
Gln Arg Arg Ala Gly Gly Val Leu Val Ala Ser His Leu Gln Ser Phe
                                165 170
Leu Glu Val Ser Tyr Arg Val Leu Arg His Leu Ala Gln Pro
```

wherein:
Xaa at position 17 is Cys, Ala, Leu, Ser, or Glu;
Xaa at position 37 is Ala or Asn;
Xaa at position 38 is Thr, or any other amino acid except Pro;
Xaa at position 39 is Tyr, Thr, or Ser;
Xaa at position 57 is Pro or Val;
Xaa at position 58 is Trp or Asn;
Xaa at position 59 is Ala or any other amino acid except Pro;
Xaa at position 60 is Pro, Thr, Asn, or Ser,
Xaa at position 61 is Leu, or any other amino acid except Pro;
Xaa at position 62 is Ser or Thr;
Xaa at position 63 is Ser or Asn;
Xaa at position 64 is Cys or any other amino acid except Pro;
Xaa at position 65 is Pro, Ser, or Thr;
Xaa at position 66 is Ser or Thr;
Xaa at position 67 is Gln or Asn;
Xaa at position 68 is Ala or any other amino acid except Pro;
Xaa at position 69 is Leu, Thr, or Ser
Xaa at position 93 is Glu or Asn
Xaa at position 94 is Gly or any other amino acid except Pro;
Xaa at position 95 is Ile, Asn, Ser, or Thr;
Xaa at position 97 is Pro, Ser, Thr, or Asn;
Xaa at position 133 is Thr or Asn;
Xaa at position 134 is Gln or any other amino acid except Pro;
Xaa at position 135 is Gly, Ser, or Thr
Xaa at position 141 is Ala or Asn;
Xaa at position 142 is Ser or any other amino acid except Pro; and
Xaa at position 143 is Ala, Ser, or Thr;

and wherein:
Xaa at positions 37, 38, and 39 constitute region 1;
Xaa at positions 58, 59, and 60 constitute region 2;
Xaa at positions 59, 60, and 61 constitute region 3;
Xaa at positions 60, 61, and 62 constitute region 4;
Xaa at positions 61, 62, and 63 constitute region 5;
Xaa at positions 62, 63, and 64 constitute region 6;
Xaa at positions 63, 64, and 65 constitute region 7;
Xaa at positions 64, 65, and 66 constitute region 8;
Xaa at positions 67, 68, and 69 constitute region 9;
Xaa at positions 93, 94, and 95 constitute region 10;
Xaa at positions 94, 95, and Ser at position 96 constitute region 11;
Xaa at positions 95, and 97, and Ser at position 96 constitute region 12;
Xaa at positions 133, 134, and 135 constitute region 13;
Xaa at positions 141, 142, and 143 constitute region 14;

and provided that at least one of regions 1 through 14 comprises the sequence Asn Xaa1 Xaa2 wherein Xaa1 is any amino acid except Pro and Xaa2 is Ser or Thr.

Thus, the heterologous fusion proteins of the present invention include analogs wherein one or any combination of two or more regions comprise the sequence Asn Xaa1 Xaa2 wherein Xaa1 is any amino acid except Pro and Xaa2 is Ser or Thr.

Preferred hyperglycosylated G-CSF analogs that make up part of the heterologous fusion proteins of the present invention, include the following:
a) G-CSF[A37N, Y39T]
b) G-CSF[P57V, W58N, P60T]
c) G-CSF[P60N, S62T]
d) G-CSF[S63N, P65T]
e) G-CSF[Q67N, L69T]
f) G-CSF[E93N, I95T]
g) G-CSF[T133N, G135T]
h) G-CSF[A141N, A143T]
i) G-CSF[A37N, Y39T, P57V, W58N, P60T]
j) G-CSF[A37N, Y39T, P60N, S62T]
k) G-CSF[A37N, Y39T, S63N, P65T]
l) G-CSF[A37N, Y39T, Q67N, L69T]
m) G-CSF[A37N, Y39T, E93N, I95T]
n) G-CSF[A37N, Y39T, T133N, G135T]
o) G-CSF[A37N, Y39T, A141N, A143T]
p) G-CSF[A37N, Y39T, P57V, W58N, P60T, S63N, P65T]
q) G-CSF[A37N, Y39T, P57V, W58N, P60T, Q67N, L69T]
r) G-CSF[A37N, Y39T, S63N, P65T, E93N, I95T]

The present invention also includes heterologous fusion proteins, which are the product of the expression in a host cell of an exogenous DNA sequence, which comprises a DNA sequence encoding a heterologous fusion protein of Formula I (described above) fused to a DNA sequence encoding human albumin or the Fc portion of an immunoglobulin.

The present invention includes an isolated nucleic acid sequence, comprising a polynucleotide encoding a heterologous fusion protein described above. Exemplary isolated nucleic acids of the present invention include isolated nucleic acid sequence comprising a hyperglycosylated G-CSF analog selected from the group consisting of:

a) SEQ ID NO:2
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTC TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC AAC CAG ACC GCC ATG CCG GCC TTC GCC TCT GCT TTC
CGG GAC GTC GGG TTG GTC TGG CGG TAC GGC CGG AAG CGG AGA CGA AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
b) SEQ ID NO:3
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
c) SEQ ID NO:4
ACC CCC CTG GGC CCT GCC AGC TCC CTG CCC CAG AGC TTC CTC CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT AAC ACC ACC AAG CTG TGC CAC CCC GAG GAG CTG GTG
CTC TTC GAC ACA TTG TGG TGG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTC CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC GCC TCT GCT TTC
CGG GAC GTC GGG TGG GTC CCA CGG TAC GGC CGG AAG CGG AGA CGA AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
d) SEQ ID NO:5
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT AAC ACT AGC AGC TGC
GAC GAG CCT GTG ACA GAC CCG TAG GGG ACC CGA TTG GAC TCC TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC GCC TCT GCT TTC
CGG GAC GTC GGG TGG GTC CCA CGG TAC GGC CGG AAG CGG AGA CGA AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
e) SEQ ID NO:6
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AAT TGC
GAC GAG CCT GTG ACA GAC CCG TAG GGG ACC CGA GGG GAC TCG TTA ACG
ACC AGC CAG GCC CTG CAG CTG GCA GGC TGC TTG AGC CAA CTC CAT AGC
TGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC GCC TCT GCT TTC
CGG GAC GTC GGG TGG GTC CCA CGG TAC GGC CGG AAG CGG AGA CGA AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
f) SEQ ID NO:7
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC GTT AAC GCT ACC CTG AGC AGC TGC
GAC GAG CCT GTG ACA GAC CCG TAG CAA TTG CGA TGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC GCC TCT GCT TTC
CGG GAC GTC GGG TGG GTC CCA CGG TAC GGC CGG AAG CGG AGA CGA AAG
CAG CGC CGG GCA GGA GGG GTC CTG GTT GCC TCC CAT CTG GAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
g) SEQ ID NO:8
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC AAC GCC ACC CAG CTG GCA GGC TGC TTG AGC CAA CTC CAT AGC
GGG TCG TTG CGG TGG GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC GCC TCT GCT TTC
CGG GAC GTC GGG TGG GTC CCA CGG TAC GGC CGG AAG CGG AGA CGA AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
h) SEQ ID NO:9
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT GCC ACC TAC AAG CTG TGC CAC CCC GAG GAG CTG GTG
CTC TTC GAC ACA CGG TGG ATG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG AAC GGG ACC TCC
CCG GAA AAG GAG ATG GTC CCC GAG GAC GTC CGG GAC TTG CCC TGG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC GCC TCT GCT TTC
CGG GAC GTC GGG TGG GTC CCA CGG TAC GGC CGG AAG CGG AGA CGA AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
i) SEQ ID NO:10
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT AAC ACC ACC AAG CTG TGC CAC CCC GAG GAG CTG GTG
CTC TTC GAC ACA TTG TGG TGG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC AAC CAG ACC GCC ATG CCG GCC TTC GCC TCT GCT TTC
CGG GAC GTC GGG TTG GTC TGG CGG TAC GGC CGG AAG CGG AGA CGA AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
j) SEQ ID NO:11
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GCC GCA GCG CTC CAG
CGG AAT CTC GTT CAC TCC TTC TAG GTC CCG CTA CCG CGT CGC GAG GTC
GAG AAG CTG TGT AAC ACC ACC AAG CTG TGC CAC CCC GAG GAG CTG GTG
CTC TTC GAC ACA TTG TGG TGG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC AAC TCT ACC TTC
CGG GAC GTC GGG TGG GTC CCA CGG TAC GGC CGG AAG TTG AGA TGG AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
k) SEQ ID NO:12
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT AAC ACC ACC AAG CTG TGC CAC CCC GAG GAG CTG GTG
CTC TTC GAC ACA TTG TGG TGG TTC GAC ACG GTG GGG CTC CTC GAC CAC
CTG CTC GGA CAC TCT CTG GGC ATC GTT AAC GCT ACC CTG AGC AGC TGC
GAC GAG CCT GTG ACA GAC CCG TAG CAA TTG CGA TGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC GCC TCT GCT TTC
CGG GAC GTC GGG TGG GTC CCA CGG TAC GGC CGG AAG CGG AGA CGA AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
l) SEQ ID NO:13
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT AAC ACC ACC AAG CTG TGC CAC CCC GAG GAG CTG GTG
CTC TTC GAC ACA TTG TGG TGG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC AAC GCC ACC CAG CTG GCA GGC TGC TTG AGC CAA CTC CAT AGC
GGG TCG TTG CGG TGG GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC GCC TCT GCT TTC
CGG GAC GTC GGG TGG GTC CCA CGG TAC GGC CGG AAG CGG AGA CGA AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
m) SEQ ID NO:14
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT AAC ACC ACC AAG CTG TGC CAC CCC GAG GAG CTG GTG
CTC TTC GAC ACA TTG TGG TGG TTC GAC ACG GTG GGG CTC CTC GAC CAC
CTG CTC GGA CAC TCT CTG GGC ATC CCC TGG GCT CCC CTG AGC AGC TGC
GAC GAG CCT GTG ACA GAC CCG TAG GGG ACC CGA GGG GAC TCG TCG ACG
CCC AGC CAG GCC CTG CAG CTG GCA GGC TGC TTG AGC CAA CTC CAT AGC
GGG TCG GTC CGG GAC GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
AAC GGT ACC GGT CCC ACC TTG GAC ACA CTG CAG CTG GAC GTC GCC GAC
TTG CCA TGG CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC GCC TCT GCT TTC
CGG GAC GTC GGG TGG GTC CCA CGG TAC GGC CGG AAG CGG AGA CGA AAG
CAG CGC CGG GCA GGA GGG GTC CTG GTT GCC TCC CAT CTG CAG AGC TTC
GTC GCG CCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
n) SEQ ID NO:15
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT AAC ACC ACC AAG CTG TGC CAC CCC GAG GAG CTG GTG
CTC TTC GAC ACA TTG TGG TGG TTC GAC ACG GTG GGG CTC CTC GAC CAC
CTG CTC GGA CAC TCT CTG GGC ATC GTT AAC GCT ACC CTG AGC AGC TGC
GAC GAG CCT GTG ACA GAC CCG TAG CAA TTG CGA TGG GAC TCG TCG ACG
CCC AGC AAC GCC ACC CAG CTG GCA GGC TGC TTG AGC CAA CTC CAT AGC
GGG TCG TTG CGG TGG GTC GAC CGT CCG ACG AAC TCG GTT GAG GTA TCG
GGC CTT TTC CTC TAC CAG GGG CTC CTG CAG GCC CTG GAA GGG ATC TCC
CCG GAA AAG GAG ATG GTC CCC GAG GAC GTC CGG GAC CTT CCC TAG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC GCC TCT GCT TTC
CGG GAC GTC GGG TGG GTC CCA CGG TAC GGC CGG AAG CGG AGA CGA AAG
CAG CGC CGG GCA GGA GGG GTC CTG GTT GCC TCC CAT CTG CAG AGC TTC
GTC GCG GCC CGT CCT CCC CAG GAC CAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG
o) SEQ ID NO:16
ACC CCC CTG GGC CCT GCC AGC TCC CTG CCC CAG AGC TTC CTG CTC AAG
TGG GGG GAC CCG GGA CGG TCG AGG GAC GGG GTC TCG AAG GAC GAG TTC
GCC TTA GAG CAA GTG AGG AAG ATC CAG GGC GAT GGC GCA GCG CTC CAG
CGG AAT CTC GTT CAC TCC TTC TAG GTC CCG CTA CCG CGT CGC GAG GTC
GAG AAG CTG TGT AAC ACC ACC AAG CTG TGC CAC CCC GAG GAG CTG GTG
CTC TTC GAC ACA TTG TGG TGG TTC GAC ACG GTG GGG CTC CTC GAC CAC
CTG CTC GGA CAC TCT CTG GGC ATC CCC TGG GCT CCC CTG AGC AAT TGC
GAC GAG CCT GTG ACA GAC CCG TAG GGG ACC CGA GGG GAC TCG TTA ACG
ACC AGC CAG GCC CTG CAG CTG GCA GGC TGC TTG AGC CAA CTC CAT AGC
TGG TCG GTC CGG GAC GTC GAC CGT CCG ACG AAC TCG GTT GAG GTA TCG
GGC CTT TTC CTC TAC CAG GGG CTC CTG CAG GCC CTG AAC GGG ACC TCC
CCG GAA AAG GAG ATG GTC CCC GAG GAC GTC CGG GAC TTG CCC TGG AGG
CCC GAG TTG GGT CCC ACC TTG GAC ACA CTG CAG CTG GAC GTC GCC GAC
GGG CTC AAC CCA GGG TGG AAC CTG TGT GAC GTC GAC CTG CAG CGG CTG
TTT GCC ACC ACC ATC TGG CAG CAG ATG GAA GAA CTG GGA ATG GCC CCT
AAA CGG TGG TGG TAG ACC GTC GTC TAC CTT CTT GAC CCT TAC CGG GGA
GCC CTG CAG CCC ACC CAG GGT GCC ATG CCG GCC TTC GCC TCT GCT TTC
CGG GAC GTC GGG TGG GTC CCA CGG TAC GGC CGG AAG CGG AGA CGA AAG
CAG CGC CGG GCA GGA GGG GTC CTG GTT GCC TCC CAT CTG CAG AGC TTC
GTC GCG GCC CGT CCT CCC CAG GAC GAA CGG AGG GTA GAC GTC TCG AAG
CTG GAG GTG TCG TAC CGC GTC TTA AGG CAC CTT GCC CAG CCC
GAC CTC CAC AGC ATG GCG CAG AAT TCC GTG GAA CGG GTC GGG

A hyperglcosylated heterologous fusion protein of the present invention also includes polynucleotides encoding the heterologous fusion protein described herein, vectors comprising these polynucleotides and host cells transfected or transformed with the vectors described herein. Also included is a process for producing a heterologous fusion protein comprising the steps of transcribing and translating a polynucleotide described herein under conditions wherein the heterologous fusion protein is expressed in detectable amounts.

The present invention encompasses a method for increasing neutrophil levels in a mammal comprising the administration of a therapeutically effective amount of a heterologous fusion protein described above. The present invention also includes the use of the heterologous fusion proteins described above for the manufacture of a medicament for the treatment of patients with insufficient circulating neutrophil levels.

The present invention also encompasses a pharmaceutical formulation adapted for the treatment of patients with insufficient neutrophil levels comprising a glycosylated protein as described above.

### BRIEF DESCRIPTION OF THE FIGURES

The invention is further illustrated with reference to the following drawings:

FIG. 1: Schematic illustrating fourteen regions in human G-CSF wherein the amino acid sequence can be mutated to create functional glycosylation sites.

FIG. 2a: IgG1 Fc amino acid sequence encompassing the hinge region, CH2 and CH3 domains.

FIG. 2b: IgG4 Fc amino acid sequence encompassing the hinge region, CH2 and CH3 domains.

FIG. 3: Human serum albumin amino acid sequence

FIG. 4: IgG1 Fc DNA sequence

FIG. 5: IgG4 Fc DNA sequence with Ser229Pro mutation.

FIG. 6: G-CSF/IgG1 Fc fusion protein

FIG. 7: G-CSF/IgG4 Fc fusion protein

FIG. 8: G-CSF/HA fusion protein.

The present invention comprises a heterologous fusion protein. As used herein, the term heterologous fusion protein means a hyperglycosylated G-CSF analog fused to human albumin, a human albumin analog, a human albumin fragment, the Fc portion of an immunoglobulin, an analog of the Fc portion of an immunoglobulin, or a fragment of the Fc portion of an immunoglobulin. The G-CSF analog may be fused directly, or fused via a peptide linker, to an albumin or Fc protein. The albumin and Fc portion may be fused to the G-CSF analogs at either terminus or at both termini. These heterologous fusion proteins are biologically active and have an increased half-life compared to native G-CSF.

### Hyperglycosylated G-CSF Analogs

Encompassed by the invention are certain hyperglycosylated analogs of G-CSF. Analogs of G-CSF refer to human G-CSF with one or more changes in the amino acid sequence which result in an increase in the number of sites for carbohydrate attachment compared with native human G-CSF expressed in animal cells in vivo. In addition, G-CSF analogs include human G-CSF wherein the O-linked glycosylation site at position 133 is replaced with an N-linked glycosylation site. Analogs are generated by site directed mutagenesis having substitution of amino acid residues creating new sites that are available for glycosylation. Analogs having a greater carbohydrate content than that found in native human G-CSF are generated by adding glycosylation sites that do not perturb the secondary, tertiary, and quaternary structure required for activity. Furthermore, because the hyperglycosylated analogs of the present invention have a larger mass and an increased negative charge compared to native G-CSF, they will not be as rapidly cleared from the circulation.

It is preferred that the G-CSF analog have 1, 2, 3, or 4 additional sites for N-glycosylation. FIG. 1 illustrates fourteen different regions that can be glycosylated with very little effect on in vitro activity. Each region may be mutated to the consensus site for N-glycosylation addition which is Asn X1 X2 wherein X1 is any amino acid except Pro and X2 is Ser or Thr. It is preferred that the X1 amino acid be any other amino acid except Trp, Asp, Glu, or Leu and it is most preferred that the X1 amino acid be the naturally occurring amino acid. The scope of the present invention includes analogs wherein a single region (1 through 14) is mutated or wherein a region is mutated in combination with one or more other regions.

Analogs having carbohydrate attached to only a single mutated site have been expressed, purified, characterized, and tested for activity. Similarly analogs with multiple glycosylation sites have been expressed, purified, characterized, and tested for activity. For example G-CSF[A37N, Y39T] is G-CSF wherein the amino acids at positions 37 and 39 have been substituted to create a glycosylation site. This site of carbohydrate attachment is illustrated as region 1 in FIG. 1. G-CSF[A37N, Y39T, P57V, W58N, P60T] is an example of a G-CSF analog wherein amino acids in region 1 and region 2 are mutated to provide two functional glycosylation sites on a single molecule (FIG. 1).

G-CSF[A37N, Y39T, P57V, W58N, P60T, Q67N, L69T] is an example of a G-CSF analog wherein the amino acids in region 1, region 2, and region 9 are mutated to provide three functional glycosylation sites on a single molecule (FIG. 1).

Native G-CSF can be used as the backbone to create the glycosylated G-CSF analogs of the present invention. In addition, the native G-CSF backbone used to create the analogs of the present invention can be modified such that substitutions in the regions defined in FIG. 1 are made in the context of a different or improved G-CSF protein. For example, native G-CSF with a Cysteine to Alanine substitution at position 17 may reduce aggregation and enhance stability and thus, can be used as the backbone used to create the glycosylated G-CSF analogs of the present invention.

In addition, Reidhaar-Olson et al., through alanine scanning mutagenesis, describe residues critical to the activity of human G-CSF. [Reidhaar-Olson et al. (1996) Biochemistry 35:9034-9041; See also Young et al. (1997) Protein Science 6:1228-1236]. Thus, the glycosylated analogs of the present invention can be modified by substituting amino acids outside the glycosylated regions described in FIG. 1.

As outlined above, amino acid substitutions in the fusion proteins of the present invention can be based on the relative similarity of the amino acid side-chain substituents, for example, their hydrophobicity, hydrophilicity, charge, size, etc. Furthermore, substitutions can be made based on secondary structure propensity. For example, a helical amino acid can be replaced with an amino acid that would preserve the helical structure. Exemplary substitutions that take various of the foregoing characteristics into consideration in order to produce conservative amino acid changes resulting in silent changes within the present peptides, etc., can be selected from other members of the class to which the naturally occurring amino acid belongs.

The present invention also encompasses G-CSF analogs wherein the O-linked glycosylation site at position 133 is mutated to serve as an N-linked glycosylation site. The N-linked carbohydrate will generally have a higher sialic acid content which will protect it from the rapid clearance mechanisms associated with native G-CSF.

The functions of a carbohydrate chain greatly depends on the structure of the attached carbohydrate moiety. Typically compounds with a higher sialic acid content will have better stability and longer half-lives in vivo. The N-linked oligosaccharides contain sialic acid in both an α2,3 and an α2,6 linkage to galactose. [Takeuchi et al. (1988) J. Biol. Chem. 263:3657]. Typically the sialic acid in the α2,3 linkage is added to galactose on the mannose α1,6 branch and the sialic acid in the α2,6 linkage is added to the galactose on the mannose α1,3 branch. The enzymes that add these sialic acids (β-galactoside α2,3 sialyltransferase and β-galactoside α2,6 sialyltransferase) are most efficient at adding sialic acid to the mannose α1,6 and mannose α1,3 branches respectively.

Tetra-antennary N-linked oligosaccharides most commonly provide four possible sites for sialic acid attachment while bi- and tri-antennary oligosaccharide chains, which can substitute for the tetra-antennary form at Asn-liked sites, commonly have at most only two or three sialic acids attached. O-linked oligosaccharides commonly provide only two sites for sialic acid attachment. Mammalian cell cultures can be screened for those cells that preferentially add teta-antennary chains to the G-CSF analogs of the present invention, thereby maximizing the number of sites for sialic acid attachment. Different types of mammalian cells also differ with respect to the transferase enzymes present and consequently the sialic acid content and type of oligosaccharide attached at each site. One way to optimize the carbohydrate content for a given G-CSF analog is to express the analog in a cell line wherein an expression plasmid containing DNA encoding a specific sialyl transferase (e.g., α2,6 sialyltransferase) is co-transfected with the G-CSF analog expression plasmid. Alternatively a host cell line may be stably transfected with a sialyltransferase cDNA and that host cell used to express the G-CSF analog of interest. Thus, it is preferable if the oligosaccharide structure and sialic acid content are optimized for each analog encompassed by the present invention.

### Heterologous Fc Fusion Proteins:

The hyperglycosylated G-CSF analogs described above can be fused directly or via a peptide linker to the Fc portion of an immunoglobulin. (See FIGS. 6-7).

Immunoglobulins are molecules containing polypeptide chains held together by disulfide bonds, typically having two light chains and two heavy chains. In each chain, one domain (V) has a variable amino acid sequence depending on the antibody specificity of the molecule. The other domains (C) have a rather constant sequence common to molecules of the same class.

As used herein, the Fc portion of an immunoglobulin has the meaning commonly given to the term in the field of immunology. Specifically, this term refers to an antibody fragment which is obtained by removing the two antigen binding regions (the Fab fragments) from the antibody. Thus, the Fc portion is formed from approximately equal sized fragments of the constant region from both heavy chains, which associate through non-covalent interactions and disulfide bonds. The Fc portion can include the hinge regions and extend through the CH2 and CH3 domains to the C-terminus of the antibody. Representative hinge regions for human and mouse immunoglobulins can be found in Antibody Engineering, A Practical Guide, Borrebaeck, C. A. K., ed., W.H. Freeman and Co., 1992, the teachings of which are herein incorporated by reference. The amino acid sequence of a representative Fc protein containing a hinge region, CH2 and CH3 domains is shown in FIGS. 2a and 2b.

There are five types of human immunoglobulin Fc regions with different effector and pharmacokinetic properties: IgG, IgA, IgM, IgD, and IgE. IgG is the most abundant immunoglobulin in serum. IgG also has the longest half-life in serum of any immunoglobulin (23 days). Unlike other immunoglobulins, IgG is efficiently recirculated following binding to an Fc receptor. There are four IgG subclasses G1, G2, G3, and G4, each of which have different effector functions. G1, G2, and G3 can bind C1q and fix complement while G4 cannot. Even though G3 is able to bind C1q more efficiently than G1, G1 is more effective at mediating complement-directed cell lysis. G2 fixes complement very inefficiently. The C1q binding site in IgG is located at the carboxy terminal region of the CH2 domain.

All IgG subclasses are capable of binding to Fc receptors (CD16, CD32, CD64) with G1 and G3 being more effective than G2 and G4. The Fc receptor-binding region of IgG is formed by residues located in both the hinge and the carboxy terminal regions of the CH2 domain.

IgA can exist both in a monomeric and dimeric form held together by a J-chain. IgA is the second most abundant Ig in serum, but it has a half-life of only 6 days. IgA has three effector functions. It binds to an IgA specific receptor on macrophages and eosinophils, which drives phagocytosis and degranulation, respectively. It can also fix complement via an unknown alternative pathway.

IgM is expressed as either a pentamer or a hexamer, both of which are held together by a J-chain. IgM has a serum half-life of 5 days. It binds weakly to C1q via a binding site located in its CH3 domain. IgD has a half-life of 3 days in serum. It is unclear what effector functions are attributable to this Ig. IgE is a monomeric Ig and has a serum half-life of 2.5 days. IgE binds to two Fc receptors which drives degranulation and results in the release of proinflammatory agents. Depending on the desired in vivo effect, the heterologous fusion proteins of the present invention may contain any of the isotypes described above or may contain mutated Fc regions wherein the complement and/or Fc receptor binding functions have been altered. For example, one embodiment of the present invention is a Ser229Pro mutation in IgG4 Fc, which reduces monomer formation. See FIG. 5.

The heterologous fusion proteins of the present invention may contain the entire Fc portion of an immunoglobulin, fragments of the Fc portion of an immunoglobulin, or analogs thereof fused to a G-CSF analog. Furthermore, the Fc portion may be fused at either terminus or at both termini.

The heterologous fusion proteins of the present invention can consist of single chain proteins or as multi-chain polypeptides. Two or more Fc fusion proteins can be produced such that they interact through disulfide bonds that naturally form between Fc regions. These multimers can be homogeneous with respect to the G-CSF analog or they may contain different G-CSF analogs fused at the N-terminus of the Fc portion of the fusion protein.

Regardless of the final structure of the fusion protein, the Fc or Fc-like region must serve to prolong the in vivo plasma half-life of the G-CSF analog compared to the native G-CSF. Furthermore, the fused G-CSF analog must retain some biological activity. Biological activity can be determined by in vitro and in vivo methods known in the art.

Since the Fc region of IgG produced by proteolysis has the same in vivo half-life as the intact IgG molecule and Fab fragments are rapidly degraded, it is believed that the relevant sequence for prolonging half-life resides in the CH2 and/or CH3 domains. Further, it has been shown in the literature that the catabolic rates of IgG variants that do not bind the high-affinity Fc receptor or C1q are indistinguishable from the rate of clearance of the parent wild-type antibody, indicating that the catabolic site is distinct from the sites involved in Fc receptor or C1q binding. [Wawrzynczak et al., (1992) Molecular Immunology 29:221]. Site-directed mutagenesis studies using a murine IgG1 Fc region suggested that the site of the IgG1 Fc region that controls the catabolic rate is located at the CH2—CH3 domain interface.

Based on these studies, Fc regions can be modified at the catabolic site to optimize the half-life of the fusion proteins. It is preferable that the Fc region used for the heterologous fusion proteins of the present invention be derived from an IgG1 (see FIG. 4) or an IgG4 Fc region. It is even more preferable that the Fc region be IgG4 or derived from IgG4. Preferably the IgG Fc region contains both the CH2 and CH3 regions including the hinge region.

### Heterologous Albumin Fusion Proteins:

The G-CSF analogs described above can be fused directly or via a peptide linker to albumin or an analog, fragment, or derivative thereof. (See FIG. 8).

Generally the albumin proteins making up part of the fusion proteins of the present invention can be derived from albumin cloned from any species. However, human albumin and fragments and analogs thereof are preferred to reduce the risk of the fusion protein being immunogenic in humans. Human serum albumin (HA) consists of a single non-glycosylated polypeptide chain of 585 amino acids with a formula molecular weight of 66,500. The amino acid sequence of human HA is shown in FIG. 3. [See Meloun, et al. (1975) FEBS Letters 58:136; Behrens, et al. (1975) Fed. Proc. 34:591; Lawn, et al. (1981) Nucleic Acids Research 9:6102-6114; Minghetti, et al. (1986) J. Biol. Chem. 261:6747]. A variety of polymorphic variants as well as analogs and fragments of albumin have been described. (See Weitkamp, et al., (1973) Ann. Hum. Genet. 37:219]. For example, in EP 322,094, the inventors disclose various shorter forms of HA. Some of these fragments include HA(1-373), HA(1-388), HA(1-389), HA(1-369), and HA(1-419) and fragments between 1-369 and 1-419. EP 399,666 discloses albumin fragments that include HA(1-177) and HA(1-200) and fragments between HA(1-177) and HA(1-200).

It is understood that the heterologous fusion proteins of the present invention include G-CSF analogs that are coupled to any albumin protein including fragments, analogs, and derivatives wherein such fusion protein is biologically active and has a longer plasma half-life than the G-CSF analog alone. Thus, the albumin portion of the fusion protein need not necessarily have a plasma half-life equal to that of native human albumin. In addition, the albumin may be fused to either terminus or both termini of the hyperglycosylated G-CSF analog. Fragments, analogs, and derivatives are known or can be generated that have longer half-lives or have half-lives intermediate to that of native human albumin and the G-CSF analog of interest.

The heterologous fusion proteins of the present invention encompass proteins having conservative amino acid substitutions in the G-CSF analog and/or the Fc or albumin portion of the fusion protein. A “conservative substitution” is the replacement of an amino acid with another amino acid that has the same net electronic charge and approximately the same size and shape. Amino acids with aliphatic or substituted aliphatic amino acid side chains have approximately the same size when the total number carbon and heteroatoms in their side chains differs by no more than about four. They have approximately the same shape when the number of branches in their side chains differs by no more than one. Amino acids with phenyl or substituted phenyl groups in their side chains are considered to have about the same size and shape. Except as otherwise specifically provided herein, conservative substitutions are preferably made with naturally occurring amino acids.

However, the term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid analogs and derivatives; naturally occurring non-proteogenic amino acids such as norleucine, β-alanine, ornithine, GABA, etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

The incorporation of non-natural amino acids, including synthetic non-native amino acids, substituted amino acids, or one or more D-amino acids into the heterologous fusion proteins of the present invention can be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore less likely to induce humoral immune responses in the whole organism.

### General Methods for Making the Heterologous Fusion Proteins of the Present Invention.

Although the heterologous fusion proteins of the present invention can be made by a variety of different methods, recombinant methods are preferred. For purposes of the present invention, as disclosed and claimed herein, the following general molecular biology terms and abbreviations are defined below. The terms and abbreviations used in this document have their normal meanings unless otherwise designated. For example, “° C.” refers to degrees Celsius; “mmol” refers to millimole or millimoles; “mg” refers to milligrams; “μg” refers to micrograms; “ml or mL” refers to milliliters; and “μl or μL” refers to microliters. Amino acids abbreviations are as set forth in 37 C.F.R. § 1.822 (b) (2) (1994).

“Base pair” or “bp” as used herein refers to DNA or RNA. The abbreviations A, C, G, and T correspond to the 5′-monophosphate forms of the deoxyribonucleosides (deoxy)adenosine, (deoxy)cytidine, (deoxy)guanosine, and thymidine, respectively, when they occur in DNA molecules. The abbreviations U, C, G, and A correspond to the 5′-monophosphate forms of the ribonucleosides uridine, cytidine, guanosine, and adenosine, respectively when they occur in RNA molecules. In double stranded DNA, base pair may refer to a partnership of A with T or C with G. In a DNA/RNA, heteroduplex base pair may refer to a partnership of A with U or C with G. (See the definition of “complementary”, infra.)

“Digestion” or “Restriction” of DNA refers to the catalytic cleavage of the DNA with a restriction enzyme that acts only at certain sequences in the DNA (“sequence-specific endonucleases”). The various restriction enzymes used herein are commercially available and their reaction conditions, cofactors, and other requirements were used as would-be known to one of ordinary skill in the art. Appropriate buffers and substrate amounts for particular restriction enzymes are specified by the manufacturer or can be readily found in the literature.

“Ligation” refers to the process of forming phosphodiester bonds between two double stranded nucleic acid fragments. Unless otherwise provided, ligation may be accomplished using known buffers and conditions with a DNA ligase, such as T4 DNA ligase.

“Plasmid” refers to an extrachromosomal (usually) self-replicating genetic element. Plasmids are generally designated by a lower case “p” followed by letters and/or numbers. The starting plasmids herein are either commercially available, publicly available on an unrestricted basis, or can be constructed from available plasmids in accordance with published procedures. In addition, equivalent plasmids to those described are known in the art and will be apparent to the ordinarily skilled artisan.

“Recombinant DNA cloning vector” as used herein refers to any autonomously replicating agent, including, but not limited to, plasmids and phages, comprising a DNA molecule to which one or more additional DNA segments can or have been added.

“Recombinant DNA expression vector” as used herein refers to any recombinant DNA cloning vector in which a promoter to control transcription of the inserted DNA has been incorporated.

“Transcription” refers to the process whereby information contained in a nucleotide sequence of DNA is transferred to a complementary RNA sequence.

“Transfection” refers to the uptake of an expression vector by a host cell whether or not any coding sequences are, in fact, expressed. Numerous methods of transfection are known to the ordinarily skilled artisan, for example, calcium phosphate co-precipitation, liposome transfection, and electroporation. Successful transfection is generally recognized when any indication of the operation of this vector occurs within the host cell.

“Transformation” refers to the introduction of DNA into an organism so that the DNA is replicable, either as an extrachromosomal element or by chromosomal integration. Methods of transforming bacterial and eukaryotic hosts are well known in the art, many of which methods, such as nuclear injection, protoplast fusion or by calcium treatment using calcium chloride are summarized in J. Sambrook, et al., Molecular Cloning: A Laboratory Manual, (1989). Generally, when introducing DNA into Yeast the term transformation is used as opposed to the term transfection.

“Translation” as used herein refers to the process whereby the genetic information of messenger RNA (mRNA) is used to specify and direct the synthesis of a polypeptide chain.

“Vector” refers to a nucleic acid compound used for the transfection and/or transformation of cells in gene manipulation bearing polynucleotide sequences corresponding to appropriate protein molecules which, when combined with appropriate control sequences, confers specific properties on the host cell to be transfected and/or transformed. Plasmids, viruses, and bacteriophage are suitable vectors. Artificial vectors are constructed by cutting and joining DNA molecules from different sources using restriction enzymes and ligases. The term “vector” as used herein includes Recombinant DNA cloning vectors and Recombinant DNA expression vectors.

“Complementary” or “Complementarity”, as used herein, refers to pairs of bases (purines and pyrimidines) that associate through hydrogen bonding in a double stranded nucleic acid. The following base pairs are complementary: guanine and cytosine; adenine and thymine; and adenine and uracil.

“Hybridization” as used herein refers to a process in which a strand of nucleic acid joins with a complementary strand through base pairing. The conditions employed in the hybridization of two non-identical, but very similar, complementary nucleic acids varies with the degree of complementarity of the two strands and the length of the strands. Such techniques and conditions are well known to practitioners in this field.

“Isolated amino acid sequence” refers to any amino acid sequence, however, constructed or synthesized, which is locationally distinct from the naturally occurring sequence.

“Isolated DNA compound” refers to any DNA sequence, however constructed or synthesized, which is locationally distinct from its natural location in genomic DNA.

“Isolated nucleic acid compound” refers to any RNA or DNA sequence, however constructed or synthesized, which is locationally distinct from its natural location.

“Primer” refers to a nucleic acid fragment which functions as an initiating substrate for enzymatic or synthetic elongation.

“Promoter” refers to a DNA sequence which directs transcription of DNA to RNA.

“Probe” refers to a nucleic acid compound or a fragment, thereof, which hybridizes with another nucleic acid compound.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while short probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature that can be used. As a result, it follows that higher relative temperatures would tend to make the reactions more stringent, while lower temperatures less so. For additional details and explanation of stringency of hybridization reactions, see Ausubel et al., Current Protocols in Molecular Biology, Wiley Interscience Publishers, 1995.

“Stringent conditions” or “high stringency conditions”, as defined herein, may be identified by those that (1) employ low ionic strength and high temperature for washing, for example, 15 mM sodium chloride/1.5 mM sodium citrate/0.1% sodium dodecyl sulfate at 50° C.; (2) employ during hybridization a denaturing agent, such as formamide, for example, 50% (v/v) formamide with 0.1% bovine serum albumin/0.1% ficoll/0.1% polyvinylpyrrolidone/50 mM sodium phosphate buffer at pH 6.5 with 750 mM sodium chloride/75 mM sodium citrate at 42° C.; or (3) employ 50% formamide, 5×SSC (750 mM sodium chloride, 75 mM sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C. with washes at 42° C. in 0.2×SSC (30 mM sodium chloride/3 mM sodium citrate) and 50% formamide at 55° C., followed by a high-stringency wash consisting of 0.1×SSC containing EDTA at 55° C.

“Moderately stringent conditions” may be identified as described by Sambrook et al. [Molecular Cloning: A Laboratory Manual, New York: Cold Spring Harbor Press, (1989)], and include the use of washing solution and hybridization conditions (e.g., temperature, ionic strength, and % SDS) less stringent than those described above. An example of moderately stringent conditions is overnight incubation at 37° C. in a solution comprising: 20% formamide, 5×SSC (750 mM sodium chloride, 75 mM sodium citrate), 50 mM sodium phosphate at pH 7.6, 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc., as necessary to accommodate factors such as probe length and the like.

“PCR” refers to the widely-known polymerase chain reaction employing a thermally-stable DNA polymerase.

“Leader sequence” refers to a sequence of amino acids which can be enzymatically or chemically removed to produce the desired polypeptide of interest.

“Secretion signal sequence” refers to a sequence of amino acids generally present at the N-terminal region of a larger polypeptide functioning to initiate association of that polypeptide with the cell membrane and secretion of that polypeptide through the cell membrane.

### Construction of DNA Encoding the Heterologous Fusion Proteins of the Present Invention:

Wild type albumin and immunoglobulin proteins can be obtained from a variety of sources. For example, these proteins can be obtained from a cDNA library prepared from tissue or cells which express the mRNA of interest at a detectable level. Libraries can be screened with probes designed using the published DNA or protein sequence for the particular protein of interest.

Screening a cDNA or genomic library with the selected probe may be conducted using standard procedures, such as described in Sambrook et al., Molecular Cloning: A Laboratory Manual, Cold Spring Harbor Laboratory Press, NY (1989). An alternative means to isolate a gene encoding an albumin or immunoglobulin protein is to use PCR methodology [Sambrook et al., supra; Dieffenbach et al., PCR Primer: A Laboratory Manual, Cold Spring Harbor Laboratory Press, NY (1995)]. PCR primers can be designed based on published sequences.

Generally the full-length wild-type sequences cloned from a particular species can serve as a template to create analogs, fragments, and derivatives that retain the ability to confer a longer plasma half-life on the G-CSF analog that is part of the fusion protein. It is preferred that the Fc and albumin portions of the heterologous fusion proteins of the present invention be derived from the native human sequence in order to reduce the risk of potential immunogenicity of the fusion protein in humans.

In particular, it is preferred that the immunoglobulin portion of a fusion protein encompassed by the present invention contain only an Fc fragment of the immunoglobulin. Depending on whether particular effector functions are desired and the structural characteristics of the fusion protein, an Fc fragment may contain the hinge region along with the CH2 and CH3 domains or some other combination thereof. These Fc fragments can be generated using PCR techniques with primers designed to hybridize to sequences corresponding to the desired ends of the fragment. Similarly, if fragments of albumin are desired, PCR primers can be designed which are complementary to internal albumin sequences. PCR primers can also be designed to create restriction enzyme sites to facilitate cloning into expression vectors.

DNA encoding human G-CSF can be obtained from a cDNA library prepared from tissue or cells which express G-CSF mRNA at a detectable level such as monocytes, macrophages, vascular endothelial cells, fibroblasts, and some human malignant and leukemic myeloblastic cells. Libraries can be screened with probes designed using the published DNA sequence for human G-CSF. [Souza L. et al. (1986) Science 232:61-65]. Screening a cDNA or genomic library with the selected probe may be conducted using standard procedures, such as described in Sambrook et al., Molecular Cloning: A Laboratory Manual, Cold Spring Harbor Laboratory Press, NY (1989). An alternative means to isolate the gene encoding human G-CSF is to use PCR methodology [Sambrook et al., supra; Dieffenbach et al., PCR Primer: A Laboratory Manual, Cold Spring Harbor Laboratory Press, NY (1995)].

The glycosylated G-CSF analogs of the present invention can be constructed by a variety of mutagenesis techniques well known in the art. Specifically, a representative number of glycosylated G-CSF analogs were constructed using mutagenic PCR from a cloned wild-type human G-CSF DNA template (Example 1).

The glycosylated G-CSF analogs of the present invention may be produced by other methods including recombinant DNA technology or well known chemical procedures, such as solution or solid-phase peptide synthesis, or semi-synthesis in solution beginning with protein fragments coupled through conventional solution methods.

Recombinant DNA methods are preferred for producing the glycosylated G-CSF analogs of the present invention. Host cells are transfected or transformed with expression or cloning vectors described herein for glycosylated G-CSF analog production and cultured in conventional nutrient media modified as appropriate for inducing promoters, selecting transformants, or amplifying the genes encoding the desired sequences (Example 2). The culture conditions, such as media, temperature, pH and the like, can be selected by the skilled artisan without undue experimentation

Physical stability is an essential feature for therapeutic formulations. The physical stability of the heterologous fusion proteins of the present invention depends on their conformational stability, the number of charged residues (pI of the protein), the ionic strength and pH of the formulation, and the protein concentration, among other possible factors. As discussed previously, the G-CSF analog portion of the heterologous fusion proteins can be successfully glycosylated and expressed such that they maintain their three dimensional structure. Because these analogs are able to fold properly in a hyperglycosylated state, they will have improved conformational and physical stability relative to wild-type G-CSF.

While wild-type G-CSF produced in mammalian cells and bacterial cells has similar activity in vivo, the mammalian cell-produced protein has increased conformational and physical stability due to the presence of a single O-linked sugar moiety present at position 133. Thus, the G-CSF analog portion of the heterologus fusion proteins, which have an increased glycosylation content compared to wild-type G-CSF produced in mammalian or bacterial cells, will have increased stability. Furthermore, it is likely that glycosylation may inhibit inter-domain interactions and consequently enhance stability by preventing inter-domain disulfide shuffling.

The gene encoding a heterologous fusion protein can be constructed by ligating DNA encoding a G-CSF analog in-frame to DNA encoding an albumin or Fc protein. The gene encoding the G-CSF analog and the gene encoding the albumin or Fc protein can also be joined in-frame via DNA encoding a linker peptide.

The in vivo function and stability of the heterologous fusion proteins of the present invention can be optimized by adding small peptide linkers to prevent potentially unwanted domain interactions. Although these linkers can potentially be any length and consist of any combination of amino acids, it is preferred that the length be no longer than necessary to prevent unwanted domain interactions and/or optimize biological activity and/or stability. Generally, the linkers should not contain amino acids with extremely bulky side chains or amino acids likely to introduce significant secondary structure. It is preferred that the linker be serine-glycine rich and be less than 30 amino acids in length. It is more preferred that the linker be no more than 20 amino acids in length. It is even more preferred that the linker be no more than 15 amino acids in length. A preferred linker contains repeats of the sequence Gly-Gly-Gly-Gly-Ser. It is preferred that there be between 2 and 6 repeats of this sequence. It is even more preferred that there be between 3 and 4 repeats of this sequence.

To construct the heterolgous G-CSF fusion proteins, the DNA encoding wild-type G-CSF, albumin, and Fc polypeptides and fragments thereof can be mutated either before ligation or in the context of a cDNA encoding an entire fusion protein. A variety of mutagenesis techniques are well known in the art. For example, a mutagenic PCR method utilizes strand overlap extension to create specific base mutations for the purposes of changing a specific amino acid sequence in the corresponding protein. This PCR mutagenesis requires the use of four primers, two in the forward orientation (primers A and C) and two in the reverse orientation (primers B and D). A mutated gene is amplified from the wild-type template in two different stages. The first reaction amplifies the gene in halves by performing an A to B reaction and a separate C to D reaction wherein the B and C primers target the area of the gene to be mutated. When aligning these primers with the target area, they contain mismatches for the bases that are targeted to be changed. Once the A to B and C to D reactions are complete, the reaction products are isolated and mixed for use as the template for the A to D reaction. This reaction then yields the full, mutated product.

Once a gene encoding an entire fusion protein is produced it can be cloned into an appropriate expression vector. Specific strategies that can be employed to make the G-CSF fusion proteins of the present invention are described in example 1.

### General Methods to Recombinantly Express the Heterologous Fusion Proteins of the Present Invention:

Host cells are transfected or transformed with expression or cloning vectors described herein for heterologous fusion protein production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook, et al., supra. Methods of transfection are known to the ordinarily skilled artisan, for example, CaPO₄ and electroporation. General aspects of mammalian cell host system transformations have been described in U.S. Pat. No. 4,399,216. Transformations into yeast are typically carried out according to the method of van Solingen et al., J Bact. 130(2): 946-7 (1977) and Hsiao et al., Proc. Natl. Acad. Sci. USA 76(8): 3829-33 (1979). Suitable host cells for the expression of the fusion proteins of the present invention are derived from multicellular organisms.

The fusion proteins of the present invention may be recombinantly produced directly, or as a protein having a signal sequence or other additional sequences which create a specific cleavage site at the N-terminus of the mature fusion protein. In general, the signal sequence may be a component of the vector, or it may be a part of the fusion protein-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces cc-factor leaders, the latter described in U.S. Pat. No. 5,010,182), or acid phosphatase leader, the C. albicans glucoamylase leader (EP 362,179), or the signal described in WO 90/13646. In mammalian cell expression, mammalian signal sequences may be used to direct secretion of the protein, such as signal sequences from secreted polypeptides of the same or related species as well as viral secretory leaders.

Both expression and cloning vectors contain a nucleic acid-sequence that enables the vector to replicate in one or more selected host cells. Such sequences are well known for a variety of bacteria, yeast, and viruses. The origin of replication from the plasmid pBR322 is suitable for most Gram-negative bacteria, the 2 u plasmid origin is suitable for yeast, and various viral origins (SV40, polyoma, adenovirus, VSV or BPV) are useful for cloning vectors in mammalian cells. Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement autotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the fusion protein-encoding nucleic acid, such as DHFR or thymidine kinase. An appropriate host cell when wild-type DHFR is employed is the CHO cell line deficient in DHFR activity, prepared and propagated as described [Urlaub and Chasin, Proc. Natl. Acad. Sci. USA, 77(7): 4216-20 (1980)]. A suitable selection gene for use in yeast is the trp1 gene present in the yeast plasmid Yrp7 [Stinchcomb, et al., Nature 282(5734): 39-43 (1979); Kingsman, et al., Gene 7(2): 141-52 (1979); Tschumper, et al., Gene 10(2): 157-66 (1980)]. The trp1 gene provides a selection marker for a mutant strain of yeast lacking the ability to grow in tryptophan, for example, ATCC No. 44076 or PEPC1 [Jones, Genetics 85: 23-33 (1977)].

Expression and cloning vectors usually contain a promoter operably linked to the fusion protein-encoding nucleic acid sequence to direct mRNA synthesis. Promoters recognized by a variety of potential host cells are well known. Promoters suitable for use with prokaryotic hosts include the β-lactamase and lactose promoter systems [Chang, et al., Nature 275(5681): 617-24 (1978); Goeddel, et al., Nature 281(5732): 544-8 (1979)], alkaline phosphatase, a tryptophan (up) promoter system [Goeddel, Nucleic Acids Res. 8(18): 4057-74 (1980); EP 36,776 published 30 Sep. 1981], and hybrid promoters such as the tat promoter [deBoer, et al., Proc. Natl. Acad. Sci. USA 80(1): 21-5 (1983)]. Promoters for use in bacterial systems also will contain a Shine-Dalgarno (S.D.) sequence operably linked to the DNA encoding the fusion protein.

Transcription of a polynucleotide encoding a fusion protein by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, a-ketoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the fusion protein coding sequence but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and occasionally 3′ untranslated regions of eukaryotic or viral DNAs or cDNAs. These regions contain nucleotide segments transcribed as polyadenylated fragments in the untranslated portion of the mRNA encoding the fusion protein.

Various forms of a fusion protein may be recovered from culture medium or from host cell lysates. If membrane-bound, it can be released from the membrane using a suitable detergent solution (e.g., Triton-X 100) or by enzymatic cleavage. Cells employed in expression of a fusion protein can be disrupted by various physical or chemical means, such as freeze-thaw cycling, sonication, mechanical disruption, or cell lysing agents.

### Purification of the Heterologous Fusion Proteins of the Present Invention:

Once the heterologous fusion proteins of the present invention are expressed in the appropriate host cell, the analogs can be isolated and purified. The following procedures are exemplary of suitable purification procedures:

Various methods of protein purification may be employed and such methods are known in the art and described, for example, in Deutscher, Methods in Enzymology 182: 83-9 (1990) and Scopes, Protein Purification: Principles and Practice, Springer-Verlag, NY (1982). The purification step(s) selected will depend on the nature of the production process used and the particular fusion protein produced. For example, fusion proteins comprising an Fc fragment can be effectively purified using a Protein A or Protein G affinity matix. Low or high pH buffers can be used to elute the fusion protein from the affinity matrix. Mild elution conditions will aid in preventing irreversible denaturation of the fusion protein. Imidazole-containing buffers can also be used. Example 3 describes some successful purification protocols for the fusion proteins of the present invention.

### Characterization of the Heterologous Fusion Proteins of the Present Invention:

Numerous methods exist to characterize the fusion proteins of the present invention. Some of these methods include: SDS-PAGE coupled with protein staining methods or immunoblotting using anti-IgG, anti-HA and anti-G-CSF antibodies. Other methods include matrix assisted laser desporption/ionization-mass spectrometry (MALDI-MS), liquid chromatography/mass spectrometry, isoelectric focusing, analytical anion exchange, chromatofocussing, and circular dichroism to name a few. A representative number of heterologous fusion proteins were characterized using SDS-PAGE coupled with immunoblotting as well as mass spectrometry

For example, Table 2 illustrates the calculated molecular mass for a representative number of fusion proteins as well as the observed mass (as measured by protease mapping/LC-MS). The relative differences between observed mass and mass calculated for a nonglycosylated protein are indicative of the extent of glycosylation.

The heterologous fusion proteins of the present invention may be formulated with one or more excipients. The active fusion proteins of the present invention may be combined with a pharmaceutically acceptable buffer, and the pH adjusted to provide acceptable stability, and a pH acceptable for administration such as parenteral administration.

Optionally, one or more pharmaceutically-acceptable anti-microbial agents may be added. Meta-cresol and phenol are preferred pharmaceutically-acceptable microbial agents. One or more pharmaceutically-acceptable salts may be added to adjust the ionic strength or tonicity. One or more excipients may be added to adjust the isotonicity of the formulation. Glycerin is an example of an isotonicity-adjusting excipient. Pharmaceutically acceptable means suitable for administration to a human or other animal and thus, does not contain toxic elements or undesirable contaminants and does not interfere with the activity of the active compounds therein.

A pharmaceutically-acceptable salt form of the heterologous fusion proteins of the present invention may be used in the present invention. Acids commonly employed to form acid addition salts are inorganic acids such as hydrochloric acid, hydrobromic acid, hydriodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Preferred acid addition salts are those formed with mineral acids such as hydrochloric acid and hydrobromic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

### Administration of Compositions:

Administration may be via any route known to be effective by the physician of ordinary skill. Peripheral, parenteral is one such method. Parenteral administration is commonly understood in the medical literature as the injection of a dosage form into the body by a sterile syringe or some other mechanical device such as an infusion pump. Peripheral parenteral routes can include intravenous, intramuscular, subcutaneous, and intraperitoneal routes of administration.

The heterologous fusion proteins of the present invention may also be amenable to administration by oral, rectal, nasal, or lower respiratory routes, which are non-parenteral routes. Of these non-parenteral routes, the lower respiratory route and the oral route are preferred.

The heterologous fusion proteins of the present invention can be used to treat patients with insufficient circulating neutrophil levels, typically those undergoing cancer chemotherapy.

An “effective amount” of the heterologous fusion protein is the quantity which results in a desired therapeutic and/or prophylactic effect without causing unacceptable side-effects when administered to a subject in need of G-CSF receptor stimulation. A “desired therapeutic effect” includes one or more of the following: 1) an amelioration-of the symptom(s) associated with the disease or condition; 2) a delay in the onset of symptoms associated with the disease or condition; 3) increased longevity compared with the absence of the treatment; and 4) greater quality of life compared with the absence of the treatment.

The present invention comprises G-CSF compounds that have improved biochemical and biophysical properties by virtue of being fused to an albumin protein, an albumin fragment, an albumin analog, a Fc protein, a Fc fragment, or a Fc analog. These heterologous proteins can be successfully expressed in host cells, retain signaling activities associated with activation of the G-CSF receptor, and have prolonged half-lives.

The following examples are presented to further describe the present invention. The scope of the present invention is not to be construed as merely consisting of the following examples. Those skilled in the art will recognize that the particular reagents, equipment, and procedures described are merely illustrative and are not intended to limit the present invention in any manner.

### EXAMPLES

### Example 1

### Construction of DNA Encoding Glycosylated G-CSF Analogs

Table 1 provides the sequence of primers used to create functional glycosylation sites in different regions of the protein (See FIG. 1).

TABLE 1
Primer sequences used to introduce mutations into
human G-CSF.

| | | | | |
|---|---|---|---|---|
| Mutation | A Primer* | B Primer* | C Primer* | D Primer* |

| | | | | |
|---|---|---|---|---|
| WT | CF177[SEQ ID | CF178[SEQ ID | CF179[SEQ ID | CF176[SEQ ID |
| | NO:25] | NO:26] | NO:27] | NO:28) |
| | [symbol] | GGGGCAGGGAGC | GGACAGTGCAGG | [symbol] |
| | [symbol] | TGGCTGGGCCCA | AAGCCACTCCAC | [symbol] |
| | [symbol] | GTGGAGTGGCTT | TGGGCCCAGCCA | CTGGGCAAGGTG |
| | GCCGGACCTGCC | CCTGCACTGTCC | GCTCCCTGCCCC | CCTTAAGACGCG |
| | ACCCAGAGCCCC | AGAGTGCACTGT | AGAGCTTCCTG | GTACGACACCTC |
| | ATGAAGCTG | G | | CAGGAAGCTCTG |
| C17A | CF177[SEQ ID | C17Arev[SEQ | C17Afor[SEQ | CF176[SEQ ID |
| SacI | NO:29] | ID NO:30] | ID NO:31] | NO:32] |
| | [symbol] | GCTCTAAGGCCT | GGGCCCAGCGAG | [symbol] |
| | [symbol] | TGAGCAGGAAGC | CTCCCTGCCCCA | [symbol] |
| | [symbol] | TCTGGGGCAGGG | GAGCTTCCTGCT | CTGGGCAAGGTG |
| | GCCGGACCTGCC | AGCTCGCTGGGC | CAAGGCCTTAGA | CCTTAAGACGCG |
| | ACCCAGAGCCCC | CCAGTGGAG | GCAAG | GTACGACACCTC |
| | ATGAAGCTG | | | CAGGAAGCTCTG |
| A37N, Y39T | CF177[SEQ ID | A37Nrev[SEQ | A37Nfor[SEQ | CF176[SEQ ID |
| SpeI | NO:33] | ID NO:34] | ID NO:35] | NO:36] |
| | [symbol] | GTCCGAGCAGCA | GGCGCAGCGCTC | [symbol] |
| | [symbol] | CTAGTTCCTCGG | CAGGAGAAGCTG | [symbol] |
| | [symbol] | GGTGGCACAGCT | TGTAACACCACC | CTGGGCAAGGTG |
| | GCCGGACCTGCC | TGGTGGTGTTAC | AAGCTGTGCCAC | CCTTAAGACGCG |
| | ACCCAGAGCCCC | ACAGCTTCTCCT | CCCGAGGAACTA | GTACGACACCTC |
| | ATGAAGCTG | G | GTGCTG | CAGGAAGCTCTG |
| T133N, | CF177[SEQ ID | T133Nrev[SEQ | T133Nfor[SEQ | CF176[SEQ ID |
| G135T | NO:37] | ID NO:38] | ID NO:39] | NO:40] |
| Eco47III | [symbol] | GCCCGGCGCTGG | GGCCCCTGCCCT | [symbol] |
| | [symbol] | AAAGCGCTGGCG | GCAGCCCAACCA | [symbol] |
| | [symbol] | AAGGCCGGCATG | GACCGCCATGCC | CTGGGCAAGGTG |
| | GCCGGACCTGCC | GCGGTCTGGTTG | GGCCTTCGCCAG | CCTTAAGACGCG |
| | ACCCAGAGCCCC | GGCTGCAGGGCA | CGCTTTCCAGCG | GTACGACACCTC |
| | ATGAAGCTG | G | | CAGGAAGCTCTG |
| A141N, | CF177[SEQ ID | A141Nrev[SEQ | A141Nfor[SEQ | CF176[SEQ ID |
| A143T | NO:41] | ID NO:42] | ID NO:43] | NO:44] |
| SapI | [symbol] | GCCCGGCGCTGG | GGGAATGGCCCC | [symbol] |
| | [symbol] | AAGGTAGAGTTG | TGCTCTTCAGCC | [symbol] |
| | [symbol] | AAGGCCGGCATG | CACCCAGGGTGC | CTGGGCAAGGTG |
| | GCCGGACCTGCC | GCACCCTGGGTG | CATGCCGGCCTT | CCTTAAGACGCG |
| | ACCCAGAGCCCC | GGCTGAAGAGCA | CAACTCTACCTT | GTACGACACCTC |
| | ATGAAGCTG | GGGGCCAT | CCAGCGCCGGGC | CAGGAAGCTCTG |
| | | | AG | |
| P57V, | JCB128[SEQ | JCB136[SEQ | JCB137[SEQ | JCB129[SEQ |
| W58N, P60T | ID NO:45] | ID NO:46] | ID NO:47] | ID NO:48] |
| HpaI | GCTAGCGGCGCG | GCTCAGGGTAGC | GGGCATCGTTAA | [symbol] |
| | CCACCATG | GTTAACGATGCC | CGCTACCCTGAG | CTCATTAGGGCT |
| | | CAGAGAGTG | CAGCTG | GGG |
| Q67N, LS9T | JCB134[SEQ | JCB138[SEQ | JCB139[SEQ | JCB135[SEQ |
| NaeI | ID NO:49] | ID NO:50] | ID NO:51] | ID NO:52] |
| | GCTAGCGGCGCG | CAAGCAGCCGGC | GCCCCAGCAACG | [symbol] |
| | CCACCATGGCCG | CAGCTGGGTGGC | CCACCCAGCTGG | CTCATTAGGGCT |
| | GACCTGCCACCC | GTTGCTGGGGCA | CCGGCTGCTTGA | GGGCAAGGTGCC |
| | AG | GCTGCTCAG | G | TTAAGACGCGG |
| P60N, S62T | JCB128[SEQ | JCB130[SEQ | JCB131[SEQ | JCB129[SEQ |
| SpeI | ID NO:53] | ID NO:54] | ID NO:55] | ID NO:56] |
| | GCTAGCGGCGCG | GGGGCAACTAGT | GCTAACCTGACT | [symbol] |
| | CCACCATG | CAGGTTAGCCCA | AGTTGCCCCAGC | CTCATTAGGGCT |
| | | GGG | CAG | GGG |
| S63N, P65T | JCB128[SEQ | JCB132[SEQ | JCB133[SEQ | JCB129[SEQ |
| MfeI | ID NO:57] | ID NO:58] | ID NO:59] | ID NO:60] |
| | GCTAGCGGCGCG | GGTGCAATTGCT | GCAATTGCACCA | [symbol] |
| | CCACCATG | CAGGGGAGCCCA | GCCAGGCCCTG | CTCATTAGGGCT |
| | | G | | GGG |
| E93N, I95T | JCB134[SEQ | JCB140[SEQ | JCB141[SEQ | JCB135[SEQ |
| BspEI | ID NO:61] | ID NO:62] | ID NO:63] | ID NO:64] |
| | GCTAGCGGCGCG | CCGGACTGGTCC | GAACGGGACCAG | [symbol] |
| | CCACCATGGCCG | CGTTCAGGGCCT | TCCGGAGTTGGG | CTCATTAGGGCT |
| | GACCTGCCACCC | GCAGGAGCCCCT | TCCCACCTTGG | GGGCAAGGTGCC |
| | AG | G | | TTAAGACGCGG |
| SalI | JCB155[SEQ | | | |
| | ID NO:65] | | | |
| | [symbol] | | | |
| | GGCGCGCCACCA | | | |
| | TGGCCGGACCTG | | | |

*Nucleotides in bold represent changes imposed in the target sequence and nucleotides in bold and italics represent flanking sequences which may add restriction sites to facilitate cloning, Kozac sequences, or stop codons.

### Preparation 1a: DNA Encoding Wild-type Human G-CSF

A strand overlapping extension PCR reaction was used to create a wild type human G-CSF construct in order to eliminate the methylation of an ApaI site. Isolated human G-CSF cDNA served as the template for these reactions. The 5′ end A primer was used to create a restriction enzyme site prior to the start of the coding region as well as to introduce a Kozac sequence (GGCGCC) 5′ of the coding leader sequence to facilitate translation in cell culture.

The A-B product was generated using primers CF177 and CF178 in a PCR reaction. Likewise, the C-D product was produced with primers CF179 and CF176. The products were isolated and combined. The combined mixture was then used as a template with primers CF177 and CF178 to create the full-length wild-type construct. [Nelson, R. M. and Long, G. C. (1989), Anal. Biochem. 180:147-151].

The full-length product was ligated into the pCR2.1-Topo vector (Invitrogen, Inc. Cat. No. K4500-40) by way of a topoisomerase TA overhang system to create pCR2.1G-CSF.

The following protocol was used for preparation of the full-length wild-type G-CSF protein as well as each of the G-CSF analogs. Approximately 5 ng of template DNA and 15 pmol of each primer was used in the initial PCR reactions. The reactions were prepared using Platinum PCR Supermix® (GibcoBRL Cat. No. 11306-016). The PCR reactions were denatured at 94° C. for 5 min and then subject to 25 cycles wherein each cycle consisted of 30 seconds at 94° C. followed by 30 seconds at 60° C. followed by 30 seconds at 72° C. A final extension was carried out for 7 minutes at 72° C. PCR fragments were isolated from agarose gels and purified using a Qiaquick® gel extraction kit (Qiagen, Cat. No. #28706). DNA was resuspended in sterile water and used for the final PCR reaction to prepare full-length product.

Preparation 1b: DNA encoding G-CSF[A37N, Y39T, P57V, W58N, P60T, Q67N, L69T] was constructed as follows:

DNA encoding G-CSF[A37N, Y39T, Q67N, L69T] was subcloned into pJB02 to create pJB02G-CSF[A37N, Y39T, Q67N, L69T] and pJB02G-CSF[A37N, Y39T, P57V, W58N, P60T] served as the template for strand overlapping expression PCR. JCB155 and JCB136 served as the A and B primers and JCB137 and JCB135 served as the C and D primers. The full-length mutated cDNA was prepared as described previously using JCB155 and JCB134 primers. The resulting full-length DNA encodes a protein with consensus N-linked glycosylation sites in region 1, region 2, and region 9 of the protein (See FIG. 1). The full-length cDNA was ligated back into pCR2.1-Topo to create pCR2.1G-CSF[A37N, Y39T, P57V, W58N, P60T, Q67N, L69T].

Preparation 1c: DNA encoding G-CSF[A37N, Y39T, S63N, P64T, E93N, I95T] was constructed as follows:

DNA encoding G-CSF[A37N, Y39T, E93N, I95T] was subcloned into pJB02 to create pJB02G-CSF[A37N, Y39T, E93N, I95T] and pJB02G-CSF[A37N, Y39T, E93N, I95T] served as the template for strand overlapping expression PCR. JCB155 and JCB132 served as the A and B primers and JCB133 and JCB135 served as the C and D primers. The full-length mutated cDNA was prepared as described previously using JCB155 and JCB135 primers. The resulting full-length DNA encodes a protein with consensus N-linked glycosylation sites in region 1, region 7, and region 10 of the protein (See FIG. 1). The full-length cDNA was ligated back into pCR2.1-Topo to create pCR2.1G-CSF[A37N, Y39T, S63N, P64T, E93N, I95T].

Preparation 1d: DNA encoding G-CSF[C17A] which is G-CSF wherein the amino acid at position 17 is substituted with Ala is constructed as follows:

The wild-type construct in the pCR2.1-Topo vector (pCR2.1G-CSF) serves as the PCR template for the C17A mutagenesis. Strand overlapping extension PCR is performed as described previously. CF177 and C17Arev serve as the A-B primers and C17Afor and CF176 serve as the C-D primers. The full-length mutated cDNA is prepared as described previously using the CF177 and CF176 primers. The B and C primers are used to mutate the DNA such that a SacI restriction site is created and the protein expressed from the full-length sequence contains an Alanine instead of a Cysteine at position 17. The full-length cDNA is ligated back into the pCR2.1-Topo vector to create pCR2.1G-CSF[C17A] wherein the sequence is confirmed. G-CSF analog encoding DNA is then cloned into the Nhe/Xho sites of mammalian expression vector pJB02 to create pJB02G-CSF[C17A].

Preparation 1e: DNA encoding G-CSF[A37N, Y39T] is constructed as follows:

Strand overlapping extension PCR is performed using pCR2.1G-CSF[C17A] as the template. Primers CF177 and A37Nrev serve as the A-B primers and CF176 and A37Nfor serve as the C-D primers. The full-length mutated cDNA is prepared as described previously using the CF177 and CF176 primers. The B and C primers contain mismatched sequences such that a SpeI site is created in the DNA and the protein expressed from the full-length sequence contains a consensus sequence for N-linked glycosylation in region 1 of the protein. The full-length cDNA is ligated back into the pCR2.1-Topo vector to create pCR2.1G-CSF[A37N, Y39T] wherein the sequence is confirmed. G-CSF analog encoding DNA is then cloned into the Nhe/Xho sites of mammalian expression vector pJB02 to create pJB02G-CSF[A37N, Y39T].

Preparation 1f: DNA encoding G-CSF[P57V, W58N, P60T] is constructed as follows:

Strand overlapping extension PCR is performed using pJB02G-CSF[C17A] as the template. Primers JCB128 and JCB136 serve as the A-B primers and JCB137 and JCB129 serve as the C-D primers. The full-length mutated cDNA is prepared as described previously using the JCB128 and JCB129 primers. The B and C primers contain mismatched sequences such that a HpaI site is created and the protein expressed from the full-length sequence contains a consensus sequence for N-linked glycosylation in region 2 of the protein. The full-length cDNA is ligated back into the pCR2.1-Topo vector to create pCR2.1G-CSF[P57V, W58N, P60T] wherein the sequence is confirmed. G-CSF analog encoding DNA is then cloned into the Nhe/Xho sites of mammalian expression vector pJB02 to create pJB02G-CSF[P57V, W58N, P60T].

Preparation 1g: DNA encoding G-CSF[P60N, S62T] is constructed as follows:

Strand overlapping extension PCR is performed using pJB02G-CSF[C17A] as the template. Primers JCB128 and JCB130 serve as the A-B primers and JCB131 and JCB129 serve as the C-D primers. The full-length mutated cDNA is prepared as described previously using the JCB128 and JCB129 primers. The B and C primers contain mismatched sequences such that a SpeI site is created and the protein expressed from the full-length sequence contains a consensus sequence for N-linked glycosylation in region 4 of the protein. The full-length cDNA is ligated back into the pCR2.1-Topo vector to create pCR2.1G-CSF[P60N, S62T] wherein the sequence is confirmed. G-CSF analog encoding DNA is then cloned into the Nhe/Xho sites of mammalian expression vector pJB02 to create pJB02G-CSF[P60N, S62T].

Preparation 1h: DNA encoding G-CSF[S63N, P65T] is constructed as follows:

Strand overlapping extension PCR is performed using pJB02G-CSF[C17A] as the template. Primers JCB128 and JCB132 serve as the A-B primers and JCB133 and JCB129 serve as the C-D primers. The full-length mutated cDNA is prepared as described previously using the JCB128 and JCB129 primers. The B and C primers contain mismatched sequences such that a MfeI site is created and the protein expressed from the full-length sequence contains a consensus sequence for N-linked glycosylation in region 7 of the protein. The full-length cDNA is ligated back into the pCR2.1-Topo vector to create pCR2.1G-CSF[S63N, P65T] wherein the sequence is confirmed. G-CSF analog encoding DNA is then cloned into the Nhe/Xho sites of mammalian expression vector pJB02 to create pJB02G-CSF[S63N, P65T].

Preparation 1i: DNA encoding G-CSF[Q67N, L69T] is constructed as follows:

Strand overlapping extension PCR is performed using pJB02G-CSF[C17A] as the template. Primers JCB134 and JCB138 serve as the A-B primers and JCB139 and JCB135 serve as the C-D primers. The full-length mutated cDNA is prepared as described previously using the JCB128 and JCB129 primers. The B and C primers contain mismatched sequences such that a NaeI site is created and the protein expressed from the full-length sequence contains a consensus sequence for N-linked glycosylation in region 9 of the protein. The full-length cDNA is ligated back into the pCR2.1-Topo vector to create pCR2.1G-CSF[Q67N, L69T] wherein the sequence is confirmed. G-CSF analog encoding DNA is then cloned into the Nhe/Xho sites of mammalian expression vector pJB02 to create pJB02G-CSF[Q67N, L69T].

Preparation 1j: DNA encoding G-CSF[E93N, I95T] is constructed as follows:

Strand overlapping extension PCR is performed using pJB02G-CSF[C17A] as the template. Primers JCB134 and JCB140 serve as the A-B primers and JCB141 and JCB135 serve as the C-D primers. The full-length mutated cDNA is prepared as described previously using the JCB128 and JCB129 primers. The B and C primers contain mismatched sequences such that a BspEI site is created and the protein expressed from the full-length sequence contains a consensus sequence for N-linked glycosylation in region 10 of the protein. The full-length cDNA is ligated back into the pCR2.1-Topo vector to create pCR2.1G-CSF[E93N, I95T] wherein the sequence is confirmed. G-CSF analog encoding DNA is then cloned into the Nhe/Xho sites of mammalian expression vector pJB02 to create pJB02G-CSF[E93T, I95T].

Preparation 1k: DNA encoding G-CSF[T133N, G135T] is constructed as follows:

Strand overlapping extension PCR is performed using pCR2.1G-CSF[C17A] as the template. Primers CF177 and T133Nrev serve as the A-B primers and T133Nfor and CF176 serve as the C-D primers. The full-length mutated cDNA is prepared as described previously using the CF177 and CF176 primers. The B and C primers contain mismatched sequences such that an Eco47III site is created and the protein expressed from the full-length sequence contains a consensus sequence for N-linked glycosylation in region 13 of the protein. The full-length cDNA is ligated back into the pCR2.1-Topo vector to create pCR2.1G-CSF[T133N, G135T] wherein the sequence is confirmed.

Preparation 1l: DNA encoding G-CSF[A141N, A143T] is constructed as follows:

Strand overlapping extension PCR is performed using pCR2.1G-CSF[C17A] as the template. Primers CF177 and A141Nrev serve as the A-B primers and A141Nfor and CF176 serve as the C-D primers. The full-length mutated cDNA is prepared as described previously using the CF177 and CF176 primers. The B and C primers contain mismatched sequences such that an SapI site is created and the protein expressed from the full-length sequence contains a consensus sequence for N-linked glycosylation in region 14 of the protein. The full-length cDNA is ligated back into the pCR2.1-Topo vector to create pCR2.1G-CSF[A141N, A143T] wherein the sequence is confirmed.

Preparation 1m: DNA encoding G-CSF[A37N, Y39T, T133N, G135T] is constructed as follows:

A 210 bp insert containing G-CSF[A37N, Y39T] is isolated from pCR2.1G-CSF[A37N, Y39T] using EcoNI. This fragment is ligated into pCR2.1G-CSF[T133N, G135T] which is prepared by cleavage with EcoNI and subsequent isolation of the vector (4359 bp) from a 210 bp fragment containing wild-type G-CSF sequences. This ligation creates pCR2.1G-CSF[A37N, Y39T, T133N, G135T]. Analog encoding DNA is then subcloned into pJB02 using NheI/XhoI to create pJB02G-CSF[A37N, Y39T, T133N, G135T].

Preparation 1n: DNA encoding G-CSF[A37N, Y39T, A141N, A143T] is constructed as follows:

A 210 bp insert containing G-CSF[A37N, Y39T] is isolated from pCR2.1G-CSF[A37N, Y39T] using EcoNI. This fragment is ligated into pCR2.1G-CSF[A141N, A143T] which is prepared by cleavage with EcoNI and subsequent isolation of the vector (4359 bp) from a 210 bp fragment containing wild-type G-CSF sequences. This ligation creates pCR2.1G-CSF[A37N, Y39T, A141N, A143T]. Analog encoding DNA is then subcloned into pJB02 (FIG. 3) using NheI/XhoI to create pJB02G-CSF[A37N, Y39T, A141N, A143T].

Preparation 1o: DNA encoding G-CSF[A37N, Y39T, P57V, W58N, P60T] is constructed as follows:

DNA encoding G-CSF[A37N, Y39T] is subcloned into pJB02 to create pJB02G-CSF[A37N, Y39T] and pJB02G-CSF[A37N, Y39T] serves as the template for strand overlapping expression PCR. JCB128 and JCB136 serve as the A and B primers and JCB137 and JCB129 serve as the C and D primers. The full-length mutated cDNA is prepared as-described previously using JCB128 and JCB129 primers. The resulting full-length DNA encodes a protein with consensus N-linked glycosylation sites in region 1 and region 2 of the protein. The full-length cDNA is ligated back into pCR2.1-Topo to create pCR2.1G-CSF[A37N, Y39T, P57V, W58N, P60T].

Preparation 1p: DNA encoding G-CSF[A37N, Y39T, Q67N, L69T] is constructed as follows:

DNA encoding G-CSF[A37N, Y39T] is subcloned into pJB02 to create pJB02G-CSF[A37N, Y39T] and pJB02G-CSF[A37N, Y39T] serves as the template for strand overlapping expression PCR. JCB134 and JCB138 serve as the A and B primers and JCB139 and JCB135 serve as the C and D primers. The full-length mutated cDNA is prepared as described previously using JCB128 and JCB129 primers. The resulting full-length DNA encodes a protein with consensus N-linked glycosylation sites in region 1 and region 9 of the protein. The full-length cDNA is ligated back into pCR2.1-Topo to create pCR2.1G-CSF[A37N, Y39T, Q67N, L69T].

Preparation 1q: DNA encoding G-CSF[A37N, Y39T, E93N, I95T] is constructed as follows:

DNA encoding G-CSF[A37N, Y39T] is subcloned into pJB02 to create pJB02G-CSF[A37N, Y39T] and pJB02G-CSF[A37N, Y39T] serves as the template for strand overlapping expression PCR. JCB134 and JCB140 serve as the A and B primers and JCB141 and JCB135 serve as the C and D primers. The full-length mutated cDNA is prepared as described previously using JCB128 and JCB129 primers. The resulting full-length DNA encodes a protein with consensus N-linked glycosylation sites in region 1 and region 10 of the protein. The full-length cDNA is ligated back into pCR2.1-Topo to create pCR2.1G-CSF[A37N, Y39T, E93N, I95T].

### Example 2

### Expression of Heterologus-fusion Proteins

2a: Expression in 293/EBNA Cells:

Each full-length DNA encoding a G-CSF analog was subcloned into the NheI/XhoI sites of mammalian expression vector pJB02 (FIG. 3). This vector contains both the Ori P and Epstein Barr virus nuclear antigen (EBNA) components which are necessary for sustained, transient expression in 293 EBNA cells. This expression plasmid contains a puromycin resistance gene expressed from the CMV promoter as well as an ampicillin resistance gene. The gene of interest is also expressed from the CMV promoter.

The transfection mixture was prepared by mixing 73 μl of the liposome transfection agent Fugene 6® (Roche Molecular Biochemicals, Cat. No. 1815-075) with 820 μl Opti-Mem® (GibcoBRL Cat. No. 31985-062). G-CSF pJB02 DNA (12 μg), prepared using a Qiagen plasmid maxiprep kit (Qiagen, Cat. No. 12163), was then added to the mixture. The mixture was incubated at room temperature for 15 minutes.

Cells were plated on 10 cm² plates in DMEM/F12 3:1 (GibcoBRL Cat. No. 93-0152DK) supplemented with 5% fetal bovine serum, 20 mM HEPES, 2 mM L-glutamine, and 50 μg/mL Geneticin such that the plates were 60% to 80% confluent by the time of the transfection. Immediately before the transfection mixture was added to the plates, fresh media was added. The mixture was then added dropwise to cells with intermittent swirling. Plates were then incubated at 37° C. in a 5% CO₂ atmosphere for 24 hours at which point the media was changed to Hybritech medium without serum. The media containing a secreted form of a glycosylated G-CSF analog was then isolated 48 hours later.

2b: Expression in CHO Cells:

The expression vector for expression in CHO-K1cells pEE14.1 is illustrated in FIG. 4. This vector includes the glutamine synthetase gene which enables selection using methionine sulfoximine. This gene includes two poly A signals at the 3′ end. G-CSF analogs are expressed from the CMV promoter which includes 5′ untranslated sequences from the hCMV-MIE gene to enhance mRNA levels and translatability. The SV40 poly A signal is cloned 3′ of the G-CSF analog DNA. The SV40 late promoter drives expression of GS minigene. This expression vector encoding the gene of interest was prepared for transfection using a QIAGEN Maxi Prep Kit (QIAGEN, Cat. No. 12362). The final DNA pellet (50-100 μg) was resuspended in 100 μl of basal formulation medium (GibcoBRL CD-CHO Medium without L-Glutamine, without thymidine, without hypoxanthine). Before each transfection, CHO-K1 cells were counted and checked for viability. A volume equal to 1×10⁷ cells was centrifuged and the cell pellet rinsed with basal formulation medium. The cells were centrifuged a second time and the final pellet resuspended in basal formulation medium (700 μl final volume).

The resuspended DNA and cells were then mixed together in a standard electroporation cuvette (Gene Pulsar Cuvette) used to support mammalian transfections, and placed on ice for five minutes. The cell/DNA mix was then electroporated in a BioRad Gene Pulsar device set at 300V/975 μF and the cuvette placed back on ice for five minutes. The cell/DNA mixed was then diluted into 20 ml of cell growth medium in a non-tissue culture treated T75 flask and incubated at 37° C./5% CO₂ for 48-72 hours.

The cells were counted, checked for viability, and plated at various cell densities in selective medium in 96 well tissue culture plates and incubated at 37° C. in a 5% CO₂ atmosphere. Selective medium is basal medium with 1×HT Supplement (GibcoBRL 10×HT Stock), 100 μg/mL Dextran Sulfate (Sigma 100 mg/ml stock), 1×GS Supplements (JRH BioSciences 50×Stock) and 25 μM MSX (Methionine Sulphoximine). The plates were monitored for colony formation and screened for glycosylated G-CSF analog production.

### Example 3

### Purification of Heterologous Fusion Proteins

### HA Fusions

The cell culture harvest was dialyzed against 20 mM Tris pH 7.4. An anion exchange column (1 ml Pharmacia HiTrap Q) was equilibrated with 20 mM Tris pH 7.4 and the dialyzed material loaded at 2 ml/min. The protein was eluted from the column using a linear gradient from 0 to 500 mM NaCl in 80 min at 1 ml/min and elution was monitored by UV absorbance at 280 mm. SDS-PAGE analysis was used to identify and pool fractions of interest. This pool was dialyzed against 25 mM sodium acetate (NaOAc) pH 5.0

A cation exchange column (1 ml Pharmacia HiTrap S column) was equilibrated with 25 mM NaOAc pH 5.0 and the dialysate was loaded at 1 ml/min. The protein was eluted from the column using a linear gradient from 0 to 500 mM NaCl in 30 min. The fractions were immediately neutralized with 1 M Tris pH 8 to a final pH of 7. SDS-PAGE gels were used to identify and pool fractions of interest.

### Fc Fusions

The cell culture harvest was dialyzed against 20 mM sodium phosphate pH 7.0. An affinity column (1 ml Pharmacia HiTrap Protein A or rProtein A) was equilibrated with 20 mM sodium phosphate pH 7.0 and the dialysate was loaded at 2 ml/min. 1 ml/min of 100 mM citric acid pH 3 was used to elute the protein. Fractions were immediately neutralized with 1M Tris pH 8 to pH 7 and peak fractions (determined by in-line OD280 monitoring) were further diluted with 20 mM sodium phosphate pH 7.0. SDS-PAGE analysis was used to identify and pool fractions of interest.

1-51. (canceled)

52. A heterologous fusion protein comprising a hyperglycosylated G-CSF analog fused to a polypeptide selected from the group consisting of
a) human albumin;
b) human albumin analogs; and
c) fragments of human albumin.

53. The heterologous fusion protein of claim 52, wherein the hyperglycosylated G-CSF analog is fused to the polypeptide via a peptide linker.

54. The heterologous fusion protein of claim 53 wherein the peptide linker is selected from the group consisting of:
a) a glycine rich peptide;
b) a peptide having the sequence [Gly-Gly-Gly-Gly-Ser]ₙ where n is 1, 2, 3, 4, or 5; and
c) a peptide having the sequence [Gly-Gly-Gly-Gly-Ser]₃.

55. The heterologous fusion protein of claim 52 wherein the hyperglycosylated G-CSF analog comprises the amino acid sequence of the formula I: [SEQ ID NO: 1]
```
(I)
1 5 10 15
Thr Pro Leu Gly Pro Ala Ser Ser Leu Pro Gln Ser Phe Leu Leu Lys
                        20 25 30
Xaa Leu Glu Gln Val Arg Lys Ile Gln Gly Asp Gly Ala Ala Leu Gln
                35 40 45
Glu Lys Leu Cys Xaa Xaa Xaa Lys Leu Cys His Pro Glu Glu Leu Val
        50 55 60
Leu Leu Gly His Ser Leu Gly Ile Xaa Xaa Xaa Xaa Xaa Xaa Xaa Xaa
65 70 75 80
Xaa Xaa Xaa Xaa Xaa Gln Leu Ala Gly Cys Leu Ser Gln Leu His Ser
                                85 90 95
Gly Leu Phe Leu Tyr Gln Gly Leu Leu Gln Ala Leu Xaa Xaa Xaa Ser
                        100 105 110
Xaa Glu Leu Gly Pro Thr Leu Asp Thr Leu Gln Leu Asp Val Ala Asp
                115 120 125
Phe Ala Thr Thr Ile Trp Gln Gln Met Glu Glu Leu Gly Met Ala Pro
        130 135 140
Ala Leu Gln Pro Xaa Xaa Xaa Ala Met Pro Ala Phe Xaa Xaa Xaa Phe
145 150 155 160
Gln Arg Arg Ala Gly Gly Val Leu Val Ala Ser His Leu Aln Ser Phe
                                165 170
Leu Glu Val Ser Tyr Arg Val Leu Arg His Leu Ala Gln Pro
```
wherein:
Xaa at position 17 is Cys, Ala, Leu, Ser, or Glu;
Xaa at position 37 is Ala or Asn;
Xaa at position 38 is Thr, or any other amino acid except Pro;
Xaa at position 39 is Tyr, Thr, or Ser;
Xaa at position 57 is Pro or Val;
Xaa at position 58 is Trp or Asn;
Xaa at position 59 is Ala or any other amino acid except Pro;
Xaa at position 60 is Pro, Thr, Asn, or Ser,
Xaa at position 61 is Leu, or any other amino acid except Pro;
Xaa at position 62 is Ser or Thr;
Xaa at position 63 is Ser or Asn;
Xaa at position 64 is Cys or any other amino acid except Pro;
Xaa at position 65 is Pro, Ser, or Thr;
Xaa at position 66 is Ser or Thr;
Xaa at position 67 is Gin or Asn;
Xaa at position 68 is Ala or any other amino acid except Pro;
Xaa at position 69 is Leu, Thr, or Ser
Xaa at position 93 is Glu or Asn
Xaa at position 94 is Gly or any other amino acid except Pro;
Xaa at position 95 is Ile, Asn, Ser, or Thr;
Xaa at position 97 is Pro, Ser, Thr, or Asn;
Xaa at position 133 is Thr or Asn;
Xaa at position 134 is Gln or any other amino acid except Pro;
Xaa at position 135 is Gly, Ser, or Thr
Xaa at position 141 is Ala or Asn;
Xaa at position 142 is Ser or any other amino acid except Pro; and
Xaa at position 143 is Ala, Ser, or Thr;

56. The heterologous fusion protein of claim 53 wherein the hyperglycosylated G-CSF analog comprises the amino acid sequence of the formula I: [SEQ ID NO: 1]
```
(I)
1 5 10 15
Thr Pro Leu Gly Pro Ala Ser Ser Leu Pro Gln Ser Phe Leu Leu Lys
                        20 25 30
Xaa Leu Glu Gln Val Arg Lys Ile Gln Gly Asp Gly Ala Ala Leu Gln
                35 40 45
Glu Lys Leu Cys Xaa Xaa Xaa Lys Leu Cys His Pro Glu Glu Leu Val
        50 55 60
Leu Leu Gly His Ser Leu Gly Ile Xaa Xaa Xaa Xaa Xaa Xaa Xaa Xaa
65 70 75 80
Xaa Xaa Xaa Xaa Xaa Gln Leu Ala Gly Cys Leu Ser Gln Leu His Ser
                                85 90 95
Gly Leu Phe Leu Tyr Gln Gly Leu Leu Gln Ala Leu Xaa Xaa Xaa Ser
                        100 105 110
Xaa Glu Leu Gly Pro Thr Leu Asp Thr Leu Gln Leu Asp Val Ala Asp
                115 120 125
Phe Ala Thr Thr Ile Trp Gln Gln Met Glu Glu Leu Gly Met Ala Pro
        130 135 140
Ala Leu Gln Pro Xaa Xaa Xaa Ala Met Pro Ala Phe Xaa Xaa Xaa Phe
145 150 155 160
Gln Arg Arg Ala Gly Gly Val Leu Val Ala Ser His Leu Aln Ser Phe
                                165 170
Leu Glu Val Ser Tyr Arg Val Leu Arg His Leu Ala Gln Pro
```
wherein:
Xaa at position 17 is Cys, Ala, Leu, Ser, or Glu;
Xaa at position 37 is Ala or Asn;
Xaa at position 38 is Thr, or any other amino acid except Pro;
Xaa at position 39 is Tyr, Thr, or Ser;
Xaa at position 57 is Pro or Val;
Xaa at position 58 is Trp or Asn;
Xaa at position 59 is Ala or any other amino acid except Pro;
Xaa at position 60 is Pro, Thr, Asn, or Ser,
Xaa at position 61 is Leu, or any other amino acid except Pro;
Xaa at position 62 is Ser or Thr;
Xaa at position 63 is Ser or Asn;
Xaa at position 64 is Cys or any other amino acid except Pro;
Xaa at position 65 is Pro, Ser, or Thr;
Xaa at position 66 is Ser or Thr;
Xaa at position 67 is Gln or Asn;
Xaa at position 68 is Ala or any other amino acid except Pro;
Xaa at position 69 is Leu, Thr, or Ser
Xaa at position 93 is Glu or Asn
Xaa at position 94 is Gly or any other amino acid except Pro;
Xaa at position 95 is Ile, Asn, Ser, or Thr;
Xaa at position 97 is Pro, Ser, Thr, or Asn;
Xaa at position 133 is Thr or Asn;
Xaa at position 134 is Gln or any other amino acid except Pro;
Xaa at position 135 is Gly, Ser, or Thr
Xaa at position 141 is Ala or Asn;
Xaa at position 142 is Ser or any other amino acid except Pro; and
Xaa at position 143 is Ala, Ser, or Thr;

57. The heterologous fusion protein of claim 54 wherein the hyperglycosylated G-CSF analog comprises the amino acid sequence of the formula I: [SEQ ID NO: 1]
```
(I)
1 5 10 15
Thr Pro Leu Gly Pro Ala Ser Ser Leu Pro Gln Ser Phe Leu Leu Lys
                        20 25 30
Xaa Leu Glu Gln Val Arg Lys Ile Gln Gly Asp Gly Ala Ala Leu Gln
                35 40 45
Glu Lys Leu Cys Xaa Xaa Xaa Lys Leu Cys His Pro Glu Glu Leu Val
        50 55 60
Leu Leu Gly His Ser Leu Gly Ile Xaa Xaa Xaa Xaa Xaa Xaa Xaa Xaa
65 70 75 80
Xaa Xaa Xaa Xaa Xaa Gln Leu Ala Gly Cys Leu Ser Gln Leu His Ser
                                85 90 95
Gly Leu Phe Leu Tyr Gln Gly Leu Leu Gln Ala Leu Xaa Xaa Xaa Ser
                        100 105 110
Xaa Glu Leu Gly Pro Thr Leu Asp Thr Leu Gln Leu Asp Val Ala Asp
                115 120 125
Phe Ala Thr Thr Ile Trp Gln Gln Met Glu Glu Leu Gly Met Ala Pro
        130 135 140
Ala Leu Gln Pro Xaa Xaa Xaa Ala Met Pro Ala Phe Xaa Xaa Xaa Phe
145 150 155 160
Gln Arg Arg Ala Gly Gly Val Leu Val Ala Ser His Leu Aln Ser Phe
                                165 170
Leu Glu Val Ser Tyr Arg Val Leu Arg His Leu Ala Gln Pro
```
wherein:
Xaa at position 17 is Cys, Ala, Leu, Ser, or Glu;
Xaa at position 37 is Ala or Asn;
Xaa at position 38 is Thr, or any other amino acid except Pro;
Xaa at position 39 is Tyr, Thr, or Ser;
Xaa at position 57 is Pro or Val;
Xaa at position 58 is Trp or Asn;
Xaa at position 59 is Ala or any other amino acid except Pro;
Xaa at position 60 is Pro, Thr, Asn, or Ser,
Xaa at position 61 is Leu, or any other amino acid except Pro;
Xaa at position 62 is Ser or Thr;
Xaa at position 63 is Ser or Asn;
Xaa at position 64 is Cys or any other amino acid except Pro;
Xaa at position 65 is Pro, Ser, or Thr;
Xaa at position 66 is Ser or Thr;
Xaa at position 67 is Gln or Asn;
Xaa at position 68 is Ala or any other amino acid except Pro;
Xaa at position 69 is Leu, Thr, or Ser
Xaa at position 93 is Glu or Asn
Xaa at position 94 is Gly or any other amino acid except Pro;
Xaa at position 95 is Ile, Asn, Ser, or Thr;
Xaa at position 97 is Pro, Ser, Thr, or Asn;
Xaa at position 133 is Thr or Asn;
Xaa at position 134 is Gln or any other amino acid except Pro;
Xaa at position 135 is Gly, Ser, or Thr
Xaa at position 141 is Ala or Asn;
Xaa at position 142 is Ser or any other amino acid except Pro; and
Xaa at position 143 is Ala, Ser, or Thr;

58. The heterologous fusion protein of claim 53 wherein any two regions of regions 1 through 14 comprise the sequence Asn Xaa1 Xaa2 wherein Xaa1 is any amino acid except Pro and Xaa2 is Ser or Thr.

59. The heterologous fusion protein of claim 53 wherein any three regions of regions 1 through 14 comprise the sequence Asn Xaa1 Xaa2 wherein Xaa1 is any amino acid except Pro and Xaa2 is Ser or Thr.

60. The heterologous fusion protein of claim 53 wherein any four regions of regions 1 through 14 comprise the sequence Asn Xaa1 Xaa2 wherein Xaa1 is any amino acid except Pro and Xaa2 is Ser or Thr.

61. The heterologous fusion protein of claim 53 wherein the hyperglycosylated G-CSF analog is selected from the group consisting of:
a) G-CSF[A37N, Y39T]
b) G-CSF[P57V, W58N, P60T]
c) G-CSF[P60N, S62T]
d) G-CSF[S63N, P65T]
e) G-CSF[Q67N, L69T]
f) G-CSF[E93N, I95T]
g) G-CSF[T133N, G135T]
h) G-CSF[A141N, A143T]
i) G-CSF[A37N, Y39T, P57V, W58N, P60T]
j) G-CSF[A37N, Y39T, P60N, S62T]
k) G-CSF[A37N, Y39T, S63N, P65T]
l) G-CSF[A37N, Y39T, Q67N, L69T]
m) G-CSF[A37N, Y39T, E93N, I95T]
n) G-CSF[A37N, Y39T, T133N, G135T]
o) G-CSF[A37N, Y39T, A141N, A143T]
p) G-CSF[A37N, Y39T, P57V, W58N, P60T, S63N, P65T]
q) G-CSF[A37N, Y39T, P57V, W58N, P60T, Q67N, L69T]
r) G-CSF[A37N, Y39T, S63N, P65T, E93N, I95T]

62. The heterologous fusion protein of claim 56 wherein any two regions of regions 1 through 14 comprise the sequence Asn Xaa1 Xaa2 wherein Xaa1 is any amino acid except Pro and Xaa2 is Ser or Thr.

63. The heterologous fusion protein of claim 56 wherein any three regions of regions 1 through 14 comprise the sequence Asn Xaa1 Xaa2 wherein Xaa1 is any amino acid except Pro and Xaa2 is Ser or Thr.

64. The heterologous fusion protein of claim 56 wherein any four regions of regions 1 through 14 comprise the sequence Asn Xaa1 Xaa2 wherein Xaa1 is any amino acid except Pro and Xaa2 is Ser or Thr.

65. The heterologous fusion protein of claim 56 wherein the hyperglycosylated G-CSF analog is selected from the group consisting of:
a) G-CSF[A37N, Y39T]
b) G-CSF[P57V, W58N, P60T]
c) G-CSF[P60N, S62T]
d) G-CSF[S63N, P65T]
e) G-CSF[Q67N, L69T]
f) G-CSF[E93N, I95T]
g) G-CSF[T133N, G135T]
h) G-CSF[A141N, A143T]
i) G-CSF[A37N, Y39T, P57V, W58N, P60T]
j) G-CSF[A37N, Y39T, P60N, S62T]
k) G-CSF[A37N, Y39T, S63N, P65T]
l) G-CSF[A37N, Y39T, Q67N, L69T]
m) G-CSF[A37N, Y39T, E93N, I95T]
n) G-CSF[A37N, Y39T, T133N, G135T]
o) G-CSF[A37N, Y39T, A141N, A143T]
p) G-CSF[A37N, Y39T, P57V, W58N, P60T, S63N, P65T]
q) G-CSF[A37N, Y39T, P57V, W58N, P60T, Q67N, L69T]
r) G-CSF[A37N, Y39T, S63N, P65T, E93N, I95T]

66. The heterologous fusion protein of claim 57 wherein any two regions of regions 1 through 14 comprise the sequence Asn Xaa1 Xaa2 wherein Xaa1 is any amino acid except Pro and Xaa2 is Ser or Thr.

67. The heterologous fusion protein of claim 57 wherein any three regions of regions 1 through 14 comprise the sequence Asn Xaa1 Xaa2 wherein Xaa1 is any amino acid except Pro and Xaa2 is Ser or Thr.

68. The heterologous fusion protein of claim 57 wherein any four regions of regions 1 through 14 comprise the sequence Asn Xaa1 Xaa2 wherein Xaa1 is any amino acid except Pro and Xaa2 is Ser or Thr.

69. The heterologous fusion protein of claim 57 wherein the hyperglycosylated G-CSF analog is selected from the group consisting of:
a) G-CSF[A37N, Y39T]
b) G-CSF[P57V, W58N, P60T]
c) G-CSF[P60N, S62T]
d) G-CSF[S63N, P65T]
e) G-CSF[Q67N, L69T]
f) G-CSF[E93N, I95T]
g) G-CSF[T133N, G135T]
h) G-CSF[A141N, A143T]
i) G-CSF[A37N, Y39T, P57V, W58N, P60T]
j) G-CSF[A37N, Y39T, P60N, S62T]
k) G-CSF[A37N, Y39T, S63N, P65T]
l) G-CSF[A37N, Y39T, Q67N, L69T]
m) G-CSF[A37N, Y39T, E93N, I95T]
n) G-CSF[A37N, Y39T, T133N, G135T]
o) G-CSF[A37N, Y39T, A141N, A143T]
p) G-CSF[A37N, Y39T, P57V, W58N, P60T, S63N, P65T]
q) G-CSF[A37N, Y39T, P57V, W58N, P60T, Q67N, L69T]
r) G-CSF[A37N, Y39T, S63N, P65T, E93N, I95T]

70. The heterologous fusion protein of claim 61, wherein the hyperglycosylated G-CSF analog is G-CSF[A37N, Y39T, P57V, W58N, P60T, Q67N, L69T].

71. The heterologous fusion protein of claim 65, wherein the hyperglycosylated G-CSF analog is G-CSF[A37N, Y39T, P57V, W58N, P60T, Q67N, L69T].

72. The heterologous fusion protein of claim 69, wherein the hyperglycosylated G-CSF analog is G-CSF[A37N, Y39T, P57V, W58N, P60T, Q67N, L69T].

73. The heterologous fusion protein of claim 61, wherein the hyperglycosylated G-CSF analog is G-CSF[A37N, Y39T, S63N, P65T, E93N, I95T].

74. The heterologous fusion protein of claim 65, wherein the hyperglycosylated G-CSF analog is G-CSF[A37N, Y39T, S63N, P65T, E93N, I95T].

75. The heterologous fusion protein of claim 69, wherein the hyperglycosylated G-CSF analog is G-CSF[A37N, Y39T, S63N, P65T, E93N, I95T].

76. A heterologous fusion protein which is the product of the expression in a host cell of an exogenous DNA sequence which comprises a DNA sequence encoding a heterologous fusion protein of claim 52.

77. A heterologous fusion protein which is the product of the expression in a host cell of an exogenous DNA sequence which comprises a DNA sequence encoding a heterologous fusion protein of claim 55.

78. A heterologous fusion protein which is the product of the expression in a host cell of an exogenous DNA sequence which comprises a DNA sequence encoding a heterologous fusion protein of claim 56.

79. A polynucleotide encoding a heterologous fusion protein of claim 52.

80. A polynucleotide encoding a heterologous fusion protein of claim 53.

81. A polynucleotide encoding a heterologous fusion protein of claim 55.

82. A polynucleotide encoding a heterologous fusion protein of claim 56.

83. A polynucleotide encoding a heterologous fusion protein of claim 57.

84. A polynucleotide which comprises a DNA sequence selected from the group consisting of:
a) SEQ ID NO:2
b) SEQ ID NO:3
c) SEQ ID NO:4
d) SEQ ID NO:5
e) SEQ ID NO:6
f) SEQ ID NO:7
g) SEQ ID NO:8
h) SEQ ID NO:9
i) SEQ ID NO:10
j) SEQ ID NO:11
k) SEQ ID NO:12
l) SEQ ID NO:13
m) SEQ ID NO:14
n) SEQ ID NO:15
o) SEQ ID NO:16 or
p) SEQ ID NO:17,

85. The polynucleotide of claim 84, wherein the DNA fused in-frame comprises SEQ ID NO: 17.

86. The heterologous fusion protein of claim 52 wherein the polypeptide is human albumin.

87. The heterologous fusion protein of claim 53 wherein the polypeptide is human albumin.

88. The heterologous fusion protein of claim 52 wherein the polypeptide is an N-terminal fragment of albumin.

89. The heterologous fusion protein of claim 53 wherein the polypeptide is an N-terminal fragment of albumin.

90. A method for increasing neutrophil levels in a mammal comprising administering a therapeutically effective amount of the heterologous fusion protein of claim 52.

91. A method for increasing neutrophil levels in a mammal comprising administering a therapeutically effective amount of the heterologous fusion protein of claim 53.

92. A method for increasing neutrophil levels in a mammal comprising administering a therapeutically effective amount of the heterologous fusion protein of claim 55.

93. A method for increasing neutrophil levels in a mammal comprising administering a therapeutically effective amount of the heterologous fusion protein of claim 56.

94. A method for increasing neutrophil levels in a mammal comprising administering a therapeutically effective amount of the heterologous fusion protein of claim 61.

95. A method for increasing neutrophil levels in a mammal comprising administering a therapeutically effective amount of the heterologous fusion protein of claim 70.

96. A method for increasing neutrophil levels in a mammal comprising administering a therapeutically effective amount of the heterologous fusion protein of claim 73.

97. A method for increasing neutrophil levels in a mammal comprising administering a therapeutically effective amount of the heterologous fusion protein of claim 86.

98. A method for increasing neutrophil levels in a mammal comprising administering a therapeutically effective amount of the heterologous fusion protein of claim 88.

99. A method of treating a patient with insufficient circulating neutrophil levels comprising administering to a patient in need thereof, an effective amount of a heterologous fusion protein of claim 52.

100. A method of treating a patient with insufficient circulating neutrophil levels comprising administering to a patient in need thereof, an effective amount of a heterologous fusion protein of claim 53.

101. A method of treating a patient with insufficient circulating neutrophil levels comprising administering to a patient in need thereof, an effective amount of a heterologous fusion protein of claim 55.

102. A method of treating a patient with insufficient circulating neutrophil levels comprising administering to a patient in need thereof, an effective amount of a heterologous fusion protein of claim 56.

103. A method of treating a patient with insufficient circulating neutrophil levels comprising administering to a patient in need thereof, an effective amount of a heterologous fusion protein of claim 61.

104. A method of treating a patient with insufficient circulating neutrophil levels comprising administering to a patient in need thereof, an effective amount of a heterologous fusion protein of claim 70.

105. A method of treating a patient with insufficient circulating neutrophil levels comprising administering to a patient in need thereof, an effective amount of a heterologous fusion protein of claim 73.

106. A method of treating a patient with insufficient circulating neutrophil levels comprising administering to a patient in need thereof, an effective amount of a heterologous fusion protein of claim 86.

107. A method of treating a patient with insufficient circulating neutrophil levels comprising administering to a patient in need thereof, an effective amount of a heterologous fusion protein of claim 88.

108. A pharmaceutical formulation adapted for the treatment of patients with insufficient neutrophil levels comprising a heterologous fusion protein of claim 52.

109. A pharmaceutical formulation adapted for the treatment of patients with insufficient neutrophil levels comprising a heterologous fusion protein of claim 53.

110. A pharmaceutical formulation adapted for the treatment of patients with insufficient neutrophil levels comprising a heterologous fusion protein of claim 55.

111. A pharmaceutical formulation adapted for the treatment of patients with insufficient neutrophil levels comprising a heterologous fusion protein of claim 56.

112. A pharmaceutical formulation adapted for the treatment of patients with insufficient neutrophil levels comprising a heterologous fusion protein of claim 61.

113. A pharmaceutical formulation adapted for the treatment of patients with insufficient neutrophil levels comprising a heterologous fusion protein of claim 70.

114. A pharmaceutical formulation adapted for the treatment of patients with insufficient neutrophil levels comprising a heterologous fusion protein of claim 73.

115. A pharmaceutical formulation adapted for the treatment of patients with insufficient neutrophil levels comprising a heterologous fusion protein of claim 86.

116. A pharmaceutical formulation adapted for the treatment of patients with insufficient neutrophil levels comprising a heterologous fusion protein of claim 88.

117. A heterologous fusion protein comprising a hyperglycosylated G-CSF analog fused to a polypeptide selected from the group consisting of:
a) the Fc portion of an immunoglobulin;
b) an analog of the Fc portion of an immunoglobulin; and
c) fragments of the Fc portion of an immunoglobulin.

118. The heterologous fusion protein of claim 117, wherein the hyperglycosylated G-CSF analog is fused to the polypeptide via a peptide linker.

119. The heterologous fusion protein of the claim 118 wherein the peptide linker is selected from the group consisting of:
a) a glycine rich peptide;
b) a peptide having the sequence [Gly-Gly-Gly-Gly-Ser]ₙ where n is 1, 2, 3, 4, or 5; and
c) a peptide having the sequence [Gly-Gly-Gly-Gly-Ser]₃.

120. The heterologous fusion protein of claim 117, wherein the hyperglycosylated G-CSF analog comprises the amino acid sequence of the formula I: [SEQ ID NO: 1]
```
(I)
1 5 10 15
Thr Pro Leu Gly Pro Ala Ser Ser Leu Pro Gln Ser Phe Leu Leu Lys
                        20 25 30
Xaa Leu Glu Gln Val Arg Lys Ile Gln Gly Asp Gly Ala Ala Leu Gln
                35 40 45
Glu Lys Leu Cys Xaa Xaa Xaa Lys Leu Cys His Pro Glu Glu Leu Val
        50 55 60
Leu Leu Gly His Ser Leu Gly Ile Xaa Xaa Xaa Xaa Xaa Xaa Xaa Xaa
65 70 75 80
Xaa Xaa Xaa Xaa Xaa Gln Leu Ala Gly Cys Leu Ser Gln Leu His Ser
                                85 90 95
Gly Leu Phe Leu Tyr Gln Gly Leu Leu Gln Ala Leu Xaa Xaa Xaa Ser
                        100 105 110
Xaa Glu Leu Gly Pro Thr Leu Asp Thr Leu Gln Leu Asp Val Ala Asp
                115 120 125
Phe Ala Thr Thr Ile Trp Gln Gln Met Glu Glu Leu Gly Met Ala Pro
        130 135 140
Ala Leu Gln Pro Xaa Xaa Xaa Ala Met Pro Ala Phe Xaa Xaa Xaa Phe
145 150 155 160
Gln Arg Arg Ala Gly Gly Val Leu Val Ala Ser His Leu Aln Ser Phe
                                165 170
Leu Glu Val Ser Tyr Arg Val Leu Arg His Leu Ala Gln Pro
```
wherein:
Xaa at position 17 is Cys, Ala, Leu, Ser, or Glu;
Xaa at position 37 is Ala or Asn;
Xaa at position 38 is Thr, or any other amino acid except Pro;
Xaa at position 39 is Tyr, Thr, or Ser;
Xaa at position 57 is Pro or Val;
Xaa at position 58 is Trp or Asn;
Xaa at position 59 is Ala or any other amino acid except Pro;
Xaa at position 60 is Pro, Thr, Asn, or Ser,
Xaa at position 61 is Leu, or any other amino acid except Pro;
Xaa at position 62 is Ser or Thr;
Xaa at position 63 is Ser or Asn;
Xaa at position 64 is Cys or any other amino acid except Pro;
Xaa at position 65 is Pro, Ser, or Thr;
Xaa at position 66 is Ser or Thr;
Xaa at position 67 is Gln or Asn;
Xaa at position 68 is Ala or any other amino acid except Pro;
Xaa at position 69 is Leu, Thr, or Ser
Xaa at position 93 is Glu or Asn
Xaa at position 94 is Gly or any other amino acid except Pro;
Xaa at position 95 is Ile, Asn, Ser, or Thr;
Xaa at position 97 is Pro, Ser, Thr, or Asn;
Xaa at position 133 is Thr or Asn;
Xaa at position 134 is Gln or any other amino acid except Pro;
Xaa at position 135 is Gly, Ser, or Thr
Xaa at position 141 is Ala or Asn;
Xaa at position 142 is Ser or any other amino acid except Pro; and
Xaa at position 143 is Ala, Ser, or Thr;

121. The heterologous fusion protein of claim 118, wherein the hyperglycosylated G-CSF analog comprises the amino acid sequence of the formula I: [SEQ ID NO:1]
```
(I)
1 5 10 15
Thr Pro Leu Gly Pro Ala Ser Ser Leu Pro Gln Ser Phe Leu Leu Lys
                        20 25 30
Xaa Leu Glu Gln Val Arg Lys Ile Gln Gly Asp Gly Ala Ala Leu Gln
                35 40 45
Glu Lys Leu Cys Xaa Xaa Xaa Lys Leu Cys His Pro Glu Glu Leu Val
        50 55 60
Leu Leu Gly His Ser Leu Gly Ile Xaa Xaa Xaa Xaa Xaa Xaa Xaa Xaa
65 70 75 80
Xaa Xaa Xaa Xaa Xaa Gln Leu Ala Gly Cys Leu Ser Gln Leu His Ser
                                85 90 95
Gly Leu Phe Leu Tyr Gln Gly Leu Leu Gln Ala Leu Xaa Xaa Xaa Ser
                        100 105 110
Xaa Glu Leu Gly Pro Thr Leu Asp Thr Leu Gln Leu Asp Val Ala Asp
                115 120 125
Phe Ala Thr Thr Ile Trp Gln Gln Met Glu Glu Leu Gly Met Ala Pro
        130 135 140
Ala Leu Gln Pro Xaa Xaa Xaa Ala Met Pro Ala Phe Xaa Xaa Xaa Phe
145 150 155 160
Gln Arg Arg Ala Gly Gly Val Leu Val Ala Ser His Leu Aln Ser Phe
                                165 170
Leu Glu Val Ser Tyr Arg Val Leu Arg His Leu Ala Gln Pro
```
wherein:
Xaa at position 17 is Cys, Ala, Leu, Ser, or Glu;
Xaa at position 37 is Ala or Asn;
Xaa at position 38 is Thr, or any other amino acid except Pro;
Xaa at position 39 is Tyr, Thr, or Ser;
Xaa at position 57 is Pro or Val;
Xaa at position 58 is Trp or Asn;
Xaa at position 59 is Ala or any other amino acid except Pro;
Xaa at position 60 is Pro, Thr, Asn, or Ser,
Xaa at position 61 is Leu, or any other amino acid except Pro;
Xaa at position 62 is Ser or Thr;
Xaa at position 63 is Ser or Asn;
Xaa at position 64 is Cys or any other amino acid except Pro;
Xaa at position 65 is Pro, Ser, or Thr;
Xaa at position 66 is Ser or Thr;
Xaa at position 67 is Gln or Asn;
Xaa at position 68 is Ala or any other amino acid except Pro;
Xaa at position 69 is Leu, Thr, or Ser
Xaa at position 93 is Glu or Asn
Xaa at position 94 is Gly or any other amino acid except Pro;
Xaa at position 95 is Ile, Asn, Ser, or Thr;
Xaa at position 97 is Pro, Ser, Thr, or Asn;
Xaa at position 133 is Thr or Asn;
Xaa at position 134 is Gln or any other amino acid except Pro;
Xaa at position 135 is Gly, Ser, or Thr
Xaa at position 141 is Ala or Asn;
Xaa at position 142 is Ser or any other amino acid except Pro; and
Xaa at position 143 is Ala, Ser, or Thr;

122. The heterologous fusion protein of claim 119, wherein the hyperglycosylated G-CSF analog comprises the amino acid sequence of the formula I: [SEQ ID NO: 1]
```
(I)
1 5 10 15
Thr Pro Leu Gly Pro Ala Ser Ser Leu Pro Gln Ser Phe Leu Leu Lys
                        20 25 30
Xaa Leu Glu Gln Val Arg Lys Ile Gln Gly Asp Gly Ala Ala Leu Gln
                35 40 45
Glu Lys Leu Cys Xaa Xaa Xaa Lys Leu Cys His Pro Glu Glu Leu Val
        50 55 60
Leu Leu Gly His Ser Leu Gly Ile Xaa Xaa Xaa Xaa Xaa Xaa Xaa Xaa
65 70 75 80
Xaa Xaa Xaa Xaa Xaa Gln Leu Ala Gly Cys Leu Ser Gln Leu His Ser
                                85 90 95
Gly Leu Phe Leu Tyr Gln Gly Leu Leu Gln Ala Leu Xaa Xaa Xaa Ser
                        100 105 110
Xaa Glu Leu Gly Pro Thr Leu Asp Thr Leu Gln Leu Asp Val Ala Asp
                115 120 125
Phe Ala Thr Thr Ile Trp Gln Gln Met Glu Glu Leu Gly Met Ala Pro
        130 135 140
Ala Leu Gln Pro Xaa Xaa Xaa Ala Met Pro Ala Phe Xaa Xaa Xaa Phe
145 150 155 160
Gln Arg Arg Ala Gly Gly Val Leu Val Ala Ser His Leu Aln Ser Phe
                                165 170
Leu Glu Val Ser Tyr Arg Val Leu Arg His Leu Ala Gln Pro
```
wherein:
Xaa at position 17 is Cys, Ala, Leu, Ser, or Glu;
Xaa at position 37 is Ala or Asn;
Xaa at position 38 is Thr, or any other amino acid except Pro;
Xaa at position 39 is Tyr, Thr, or Ser;
Xaa at position 57 is Pro or Val;
Xaa at position 58 is Trp or Asn;
Xaa at position 59 is Ala or any other amino acid except Pro;
Xaa at position 60 is Pro, Thr, Asn, or Ser,
Xaa at position 61 is Leu, or any other amino acid except Pro;
Xaa at position 62 is Ser or Thr;
Xaa at position 63 is Ser or Asn;
Xaa at position 64 is Cys or any other amino acid except Pro;
Xaa at position 65 is Pro, Ser, or Thr;
Xaa at position 66 is Ser or Thr;
Xaa at position 67 is Gln or Asn;
Xaa at position 68 is Ala or any other amino acid except Pro;
Xaa at position 69 is Leu, Thr, or Ser
Xaa at position 93 is Glu or Asn
Xaa at position 94 is Gly or any other amino acid except Pro;
Xaa at position 95 is Ile, Asn, Ser, or Thr;
Xaa at position 97 is Pro, Ser, Thr, or Asn;
Xaa at position 133 is Thr or Asn;
Xaa at position 134 is Gln or any other amino acid except Pro;
Xaa at position 135 is Gly, Ser, or Thr
Xaa at position 141 is Ala or Asn;
Xaa at position 142 is Ser or any other amino acid except Pro; and
Xaa at position 143 is Ala, Ser, or Thr;

123. The heterologous fusion protein of claim 120 wherein any two regions of regions 1 through 14 comprise the sequence Asn Xaa1 Xaa2 wherein Xaa1 is any amino acid except Pro and Xaa2 is Ser or Thr.

124. The heterologous fusion protein of claim 120 wherein any three regions of regions 1 through 14 comprise the sequence Asn Xaa1 Xaa2 wherein Xaa1 is any amino acid except Pro and Xaa2 is Ser or Thr.

125. The heterologous fusion protein of claim 120 wherein any four regions of regions 1 through 14 comprise the sequence Asn Xaa1 Xaa2 wherein Xaa1 is any amino acid except Pro and Xaa2 is Ser or Thr.

126. The heterologous fusion protein of claim 120 wherein the hyperglycosylated G-CSF analog is selected from the group consisting of:
a) G-CSF[A37N, Y39T]
b) G-CSF[P57V, W58N, P60T]
c) G-CSF[P60N, S62T]
d) G-CSF[S63N, P65T]
e) G-CSF[Q67N, L69T]
f) G-CSF[E93N, I95T]
g) G-CSF[T133N, G135T]
h) G-CSF[A141N, A143T]
i) G-CSF[A37N, Y39T, P57V, W58N, P60T]
j) G-CSF[A37N, Y39T, P60N, S62T]
k) G-CSF[A37N, Y39T, S63N, P65T]
l) G-CSF[A37N, Y39T, Q67N, L69T]
m) G-CSF[A37N, Y39T, E93N, I95T]
n) G-CSF[A37N, Y39T, T133N, G135T]
o) G-CSF[A37N, Y39T, A141N, A143T]
p) G-CSF[A37N, Y39T, P57V, W58N, P60T, S63N, P65T]
q) G-CSF[A37N, Y39T, P57V, W58N, P60T, Q67N, L69T]
r) G-CSF[A37N, Y39T, S63N, P65T, E93N, I95T]

127. The heterologous fusion protein of claim 121 wherein any two regions of regions 1 through 14 comprise the sequence Asn Xaa1 Xaa2 wherein Xaa1 is any amino acid except Pro and Xaa2 is Ser or Thr.

128. The heterologous fusion protein of claim 121 wherein any three regions of regions 1 through 14 comprise the sequence Asn Xaa1 Xaa2 wherein Xaa1 is any amino acid except Pro and Xaa2 is Ser or Thr.

129. The heterologous fusion protein of claim 121 wherein any four regions of regions 1 through 14 comprise the sequence Asn Xaa1 Xaa2 wherein Xaa1 is any amino acid except Pro and Xaa2 is Ser or Thr.

130. The heterologous fusion protein of claim 121 wherein the hyperglycosylated G-CSF analog is selected from the group consisting of:
a) G-CSF[A37N, Y39T]
b) G-CSF[P57V, W58N, P60T]
c) G-CSF[P60N, S62T]
d) G-CSF[S63N, P65T]
e) G-CSF[Q67N, L69T]
f) G-CSF[E93N, I95T]
g) G-CSF[T133N, G135T]
h) G-CSF[A141N, A143T]
i) G-CSF[A37N, Y39T, P57V, W58N, P60T]
j) G-CSF[A37N, Y39T, P60N, S62T]
k) G-CSF[A37N, Y39T, S63N, P65T]
l) G-CSF[A37N, Y39T, Q67N, L69T]
m) G-CSF[A37N, Y39T, E93N, I95T]
n) G-CSF[A37N, Y39T, T133N, G135T]
o) G-CSF[A37N, Y39T, A141N, A143T]
p) G-CSF[A37N, Y39T, P57V, W58N, P60T, S63N, P65T]
q) G-CSF[A37N, Y39T, P57V, W58N, P60T, Q67N, L69T]
r) G-CSF[A37N, Y39T, S63N, P65T, E93N, I95T]

131. The heterologous fusion protein of claim 122 wherein any two regions of regions 1 through 14 comprise the sequence Asn Xaa1 Xaa2 wherein Xaa1 is any amino acid except Pro and Xaa2 is Ser or Thr.

132. The heterologous fusion protein of claim 122 wherein any three regions of regions 1 through 14 comprise the sequence Asn Xaa1 Xaa2 wherein Xaa1 is any amino acid except Pro and Xaa2 is Ser or Thr.

133. The heterologous fusion protein of claim 122 wherein any four regions of regions 1 through 14 comprise the sequence Asn Xaa1 Xaa2 wherein Xaa1 is any amino acid except Pro and Xaa2 is Ser or Thr.

134. The heterologous fusion protein of claim 122 wherein the hyperglycosylated G-CSF analog is selected from the group consisting of:
a) G-CSF[A37N, Y39T]
b) G-CSF[P57V, W58N, P60T]
c) G-CSF[P60N, S62T]
d) G-CSF[S63N, P65T]
e) G-CSF[Q67N, L69T]
f) G-CSF[E93N, I95T]
g) G-CSF[T133N, G135T]
h) G-CSF[A141N, A143T]
i) G-CSF[A37N, Y39T, P57V, W58N, P60T]
j) G-CSF[A37N, Y39T, P60N, S62T]
k) G-CSF[A37N, Y39T, S63N, P65T]
l) G-CSF[A37N, Y39T, Q67N, L69T]
m) G-CSF[A37N, Y39T, E93N, I95T]
n) G-CSF[A37N, Y39T, T133N, G135T]
o) G-CSF[A37N, Y39T, A141N, A143T]
p) G-CSF[A37N, Y39T, P57V, W58N, P60T, S63N, P65T]
q) G-CSF[A37N, Y39T, P57V, W58N, P60T, Q67N, L69T]
r) G-CSF[A37N, Y39T, S63N, P65T, E93N, I95T]

135. The heterologous fusion protein of claim 126 wherein the hyperglycosylated G-CSF analog is G-CSF[A37N, Y39T, P57V, W58N, P60T, Q67N, L69T]

136. The heterologous fusion protein of claim 126 wherein the hyperglycosylated G-CSF analog is G-CSF[A37N, Y39T, S63N, P65T, E93N, I95T]

137. The heterologous fusion protein of claim 117 wherein the polypeptide is the Fc portion of an Ig selected from the group consisting of: IgG1, IgG2, IgG3, IgG4, IgE, IgA, IgD, or IgM.

138. The heterologous fusion protein of claim 118 wherein the polypeptide is the Fc portion of an Ig selected from the group consisting of: IgG1, IgG2, IgG3, IgG4, IgE, IgA, IgD, or IgM.

139. The heterologous fusion protein of claim 137 wherein the polypeptide is the Fc portion of an Ig selected from the group consisting of: IgG1, IgG2, IgG3, and IgG4.

140. The heterologous fusion protein of claim 138 wherein the polypeptide is the Fc portion of an Ig selected from the group consisting of: IgG1, IgG2, IgG3, and IgG4.

141. The heterologous fusion protein of claim 139 wherein the polypeptide is the Fc portion of an IgG1 immunoglobulin.

142. The heterologous fusion protein of claim 140 wherein the polypeptide is the Fc portion of an IgG1 immunoglobulin.

143. The heterologous fusion protein of claim 139 wherein the polypeptide is the Fc portion of an IgG4 immunoglobulin.

144. The heterologous fusion protein of claim 142 wherein the polypeptide is the Fc portion of an IgG1 immunoglobulin.

145. The heterologous fusion protein of claim 117 wherein the Fc portion is a human IgG protein.

146. The heterologous fusion protein of claim 118 wherein the Fc portion is a human IgG protein.

147. The heterologous fusion protein of claim 117 wherein the Fc portion comprises hinge, CH2, and CH3 domains.

148. The heterologous fusion protein of claim 118 wherein the Fc portion comprises hinge, CH2, and CH3 domains.

149. The heterologous fusion protein of claim 141 wherein the polypeptide has the sequence of SEQ ID NO: 33.

150. The heterologous fusion protein of claim 142 wherein the polypeptide has the sequence of SEQ ID NO: 33.

151. A polynucleotide encoding a heterologous fusion protein of claim 141, wherein the polynucleotide comprises SEQ ID NO: 22.

152. A polynucleotide encoding a heterologous fusion protein of claim 142, wherein the polynucleotide comprises SEQ ID NO: 22.

153. A polynucleotide encoding a heterologous fusion protein of claim 117.

154. A polynucleotide encoding a heterologous fusion protein of claim 118.

155. A polynucleotide encoding a heterologous fusion protein of claim 119.

156. A polynucleotide encoding a heterologous fusion protein of claim 120.

157. A polynucleotide encoding a heterologous fusion protein of claim 126.

158. A polynucleotide encoding a heterologous fusion protein of claim 135.

159. A polynucleotide encoding a heterologous fusion protein of claim 136.

160. A polynucleotide encoding a heterologous fusion protein of claim 137.

161. A vector comprising the polynucleotide of claim 79.

162. A vector comprising the polynucleotide of claim 80.

163. A vector comprising the polynucleotide of claim 81.

164. A vector comprising the polynucleotide of claim 82.

165. A vector comprising the polynucleotide of claim 83.

166. A vector comprising the polynucleotide of claim 84.

167. A vector comprising the polynucleotide of claim 85.

168. A vector comprising the polynucleotide of claim 151.

169. A vector comprising the polynucleotide of claim 153.

170. A host cell comprising the vector of claim 161.

171. A host cell comprising the vector of claim 162.

172. A host cell comprising the vector of claim 163.

173. A host cell comprising the vector of claim 164.

174. A host cell comprising the vector of claim 165.

175. A host cell comprising the vector of claim 166.

176. A host cell comprising the vector of claim 167.

177. A host cell comprising the vector of claim 168.

178. A host cell comprising the vector of claim 169.

179. A host cell expressing at least one heterologous fusion protein of claim 52.

180. A host cell expressing at least one heterologous fusion protein of claim 53.

181. A host cell expressing at least one heterologous fusion protein of claim 55.

182. A host cell expressing at least one heterologous fusion protein of claim 56.

183. A host cell expressing at least one heterologous fusion protein of claim 61.

184. A host cell expressing at least one heterologous fusion protein of claim 70.

185. A host cell expressing at least one heterologous fusion protein of claim 73.

186. A host cell expressing at least one heterologous fusion protein of claim 86.

187. A host cell expressing at least one heterologous fusion protein of claim 88.

188. A host cell expressing at least one heterologous fusion protein of claim 117.

189. A host cell expressing at least one heterologous fusion protein of claim 118.

190. A host cell expressing at least one heterologous fusion protein of claim 119.

191. A host cell expressing at least one heterologous fusion protein of claim 120.

192. A host cell expressing at least one heterologous fusion protein of claim 126.

193. A host cell expressing at least one heterologous fusion protein of claim 135.

194. A host cell expressing at least one heterologous fusion protein of claim 136.

195. A host cell expressing at least one heterologous fusion protein of claim 137.

196. The host cell of claim 179 wherein said host cell is a CHO cell.

197. The host cell of claim 180 wherein said host cell is a CHO cell.

198. The host cell of claim 181 wherein said host cell is a CHO cell.

199. The host cell of claim 182 wherein said host cell is a CHO cell.

200. The host cell of claim 183 wherein said host cell is a CHO cell.

201. The host cell of claim 184 wherein said host cell is a CHO cell.

202. The host cell of claim 185 wherein said host cell is a CHO cell.

203. The host cell of claim 186 wherein said host cell is a CHO cell.

204. The host cell of claim 187 wherein said host cell is a CHO cell.

205. The host cell of claim 188 wherein said host cell is a CHO cell.

206. The host cell of claim 189 wherein said host cell is a CHO cell.

207. The host cell of claim 190 wherein said host cell is a CHO cell.

208. The host cell of claim 191 wherein said host cell is a CHO cell.

209. The host cell of claim 192 wherein said host cell is a CHO cell.

210. The host cell of claim 193 wherein said host cell is a CHO cell.

211. The host cell of claim 194 wherein said host cell is a CHO cell.

212. The host cell of claim 195 wherein said host cell is a CHO cell.

213. A process for producing a heterologous fusion protein comprising the steps of transcribing and translating a polynucleotide of claim 117 under conditions wherein the heterologous fusion protein is expressed in detectable amounts.

214. A method for increasing neutrophil levels in a mammal comprising the administration of a therapeutically effective amount of the heterologous fusion protein of claim 124.

215. A method for increasing neutrophil levels in a mammal comprising the administration of a therapeutically effective amount of the heterologous fusion protein of claim 125.

216. A method for increasing neutrophil levels in a mammal comprising the administration of a therapeutically effective amount of the heterologous fusion protein of claim 126.

217. A method for increasing neutrophil levels in a mammal comprising the administration of a therapeutically effective amount of the heterologous fusion protein of claim 137.

218. Use of a heterologous fusion protein of claim 117 for the treatment of patients with insufficient circulating neutrophil levels.

219. A pharmaceutical formulation adapted for the treatment of patients with insufficient neutrophil levels comprising a heterologous fusion protein of claim 117.


SEQUENCE LISTING (66 sequences)

SEQ ID NO: 1 - protein, 174 aa, Artificial Sequence
  synthetic construct
TPLGPASSLPQSFLLKXLEQVRKIQGDGAALQEKLCXXXKLCHPEELVLLGHSLGIXXXXXXXXXXXXXQLAGCLSQLHSGLFLYQGLLQALXXXSXELGPTLDTLQLDVADFATTIWQQMEELGMAPALQPXXXAMPAFXXXFQRRAGGVLVASHLQSFLEVSYRVLRHLAQP

SEQ ID NO: 2 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtgccacctacaagctgtgccaccccgaggagctggtgctcttcgacacacggtggatgttcgacacggtggggctcctcgaccacctgctcggacactctctgggcatcccctgggctcccctgagcagctgcgacgagcctgtgacagacccgtaggggacccgaggggactcgtcgacgcccagccaggccctgcagctggcaggctgcttgagccaactccatagcgggtcggtccgggacgtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccaaccagaccgccatgccggccttcgcctctgctttccgggacgtcgggttggtctggcggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 3 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtgccacctacaagctgtgccaccccgaggagctggtgctcttcgacacacggtggatgttcgacacggtggggctcctcgaccacctgctcggacactctctgggcatcccctgggctcccctgagcagctgcgacgagcctgtgacagacccgtaggggacccgaggggactcgtcgacgcccagccaggccctgcagctggcaggctgcttgagccaactccatagcgggtcggtccgggacgtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcaactctaccttccgggacgtcgggtgggtcccacggtacggccggaagttgagatggaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 4 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtaacaccaccaagctgtgccaccccgaggagctggtgctcttcgacacattgtggtggttcgacacggtggggctcctcgaccacctgctcggacactctctgggcatcccctgggctcccctgagcagctgcgacgagcctgtgacagacccgtaggggacccgaggggactcgtcgacgcccagccaggccctgcagctggcaggctgcttgagccaactccatagcgggtcggtccgggacgtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcgcctctgctttccgggacgtcgggtgggtcccacggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 5 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtgccacctacaagctgtgccaccccgaggagctggtgctcttcgacacacggtggatgttcgacacggtggggctcctcgaccacctgctcggacactctctgggcatcccctgggctaacactagcagctgcgacgagcctgtgacagacccgtaggggacccgattggactcctcgacgcccagccaggccctgcagctggcaggctgcttgagccaactccatagcgggtcggtccgggacgtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcgcctctgctttccgggacgtcgggtgggtcccacggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 6 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtgccacctacaagctgtgccaccccgaggagctggtgctcttcgacacacggtggatgttcgacacggtggggctcctcgaccacctgctcggacactctctgggcatcccctgggctcccctgagcaattgcgacgagcctgtgacagacccgtaggggacccgaggggactcgttaacgaccagccaggccctgcagctggcaggctgcttgagccaactccatagctggtcggtccgggacgtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcgcctctgctttccgggacgtcgggtgggtcccacggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 7 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtgccacctacaagctgtgccaccccgaggagctggtgctcttcgacacacggtggatgttcgacacggtggggctcctcgaccacctgctcggacactctctgggcatcgttaacgctaccctgagcagctgcgacgagcctgtgacagacccgtagcaattgcgatgggactcgtcgacgcccagccaggccctgcagctggcaggctgcttgagccaactccatagcgggtcggtccgggacgtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcgcctctgctttccgggacgtcgggtgggtcccacggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 8 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtgccacctacaagctgtgccaccccgaggagctggtgctcttcgacacacggtggatgttcgacacggtggggctcctcgaccacctgctcggacactctctgggcatcccctgggctcccctgagcagctgcgacgagcctgtgacagacccgtaggggacccgaggggactcgtcgacgcccagcaacgccacccagctggcaggctgcttgagccaactccatagcgggtcgttgcggtgggtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcgcctctgctttccgggacgtcgggtgggtcccacggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 9 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtgccacctacaagctgtgccaccccgaggagctggtgctcttcgacacacggtggatgttcgacacggtggggctcctcgaccacctgctcggacactctctgggcatcccctgggctcccctgagcagctgcgacgagcctgtgacagacccgtaggggacccgaggggactcgtcgacgcccagccaggccctgcagctggcaggctgcttgagccaactccatagcgggtcggtccgggacgtcgaccgtccgacgaactcggttgaggtatcgggccttttcctctaccaggggctcctgcaggccctgaacgggacctccccggaaaaggagatggtccccgaggacgtccgggacttgccctggaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcgcctctgctttccgggacgtcgggtgggtcccacggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 10 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtaacaccaccaagctgtgccaccccgaggagctggtgctcttcgacacattgtggtggttcgacacggtggggctcctcgaccacctgctcggacactctctgggcatcccctgggctcccctgagcagctgcgacgagcctgtgacagacccgtaggggacccgaggggactcgtcgacgcccagccaggccctgcagctggcaggctgcttgagccaactccatagcgggtcggtccgggacgtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccaaccagaccgccatgccggccttcgcctctgctttccgggacgtcgggttggtctggcggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 11 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtaacaccaccaagctgtgccaccccgaggagctggtgctcttcgacacattgtggtggttcgacacggtggggctcctcgaccacctgctcggacactctctgggcatcccctgggctcccctgagcagctgcgacgagcctgtgacagacccgtaggggacccgaggggactcgtcgacgcccagccaggccctgcagctggcaggctgcttgagccaactccatagcgggtcggtccgggacgtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcaactctaccttccgggacgtcgggtgggtcccacggtacggccggaagttgagatggaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 12 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtaacaccaccaagctgtgccaccccgaggagctggtgctcttcgacacattgtggtggttcgacacggtggggctcctcgaccacctgctcggacactctctgggcatcgttaacgctaccctgagcagctgcgacgagcctgtgacagacccgtagcaattgcgatgggactcgtcgacgcccagccaggccctgcagctggcaggctgcttgagccaactccatagcgggtcggtccgggacgtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcgcctctgctttccgggacgtcgggtgggtcccacggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 13 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtaacaccaccaagctgtgccaccccgaggagctggtgctcttcgacacattgtggtggttcgacacggtggggctcctcgaccacctgctcggacactctctgggcatcccctgggctcccctgagcagctgcgacgagcctgtgacagacccgtaggggacccgaggggactcgtcgacgcccagcaacgccacccagctggcaggctgcttgagccaactccatagcgggtcgttgcggtgggtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcgcctctgctttccgggacgtcgggtgggtcccacggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 14 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtaacaccaccaagctgtgccaccccgaggagctggtgctcttcgacacattgtggtggttcgacacggtggggctcctcgaccacctgctcggacactctctgggcatcccctgggctcccctgagcagctgcgacgagcctgtgacagacccgtaggggacccgaggggactcgtcgacgcccagccaggccctgcagctggcaggctgcttgagccaactccatagcgggtcggtccgggacgtcgaccgtccgacgaactcggttgaggtatcgggccttttcctctaccaggggctcctgcaggccctggaagggatctccccggaaaaggagatggtccccgaggacgtccgggaccttccctagaggaacggtaccggtcccaccttggacacactgcagctggacgtcgccgacttgccatggccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcgcctctgctttccgggacgtcgggtgggtcccacggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 15 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtaacaccaccaagctgtgccaccccgaggagctggtgctcttcgacacattgtggtggttcgacacggtggggctcctcgaccacctgctcggacactctctgggcatcgttaacgctaccctgagcagctgcgacgagcctgtgacagacccgtagcaattgcgatgggactcgtcgacgcccagcaacgccacccagctggcaggctgcttgagccaactccatagcgggtcgttgcggtgggtcgaccgtccgacgaactcggttgaggtatcgggccttttcctctaccaggggctcctgcaggccctggaagggatctccccggaaaaggagatggtccccgaggacgtccgggaccttccctagaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcgcctctgctttccgggacgtcgggtgggtcccacggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 16 - DNA, 1044 bp, Artificial Sequence
  synthetic construct
acccccctgggccctgccagctccctgccccagagcttcctgctcaagtggggggacccgggacggtcgagggacggggtctcgaaggacgagttcgccttagagcaagtgaggaagatccagggcgatggcgcagcgctccagcggaatctcgttcactccttctaggtcccgctaccgcgtcgcgaggtcgagaagctgtgtaacaccaccaagctgtgccaccccgaggagctggtgctcttcgacacattgtggtggttcgacacggtggggctcctcgaccacctgctcggacactctctgggcatcccctgggctcccctgagcaattgcgacgagcctgtgacagacccgtaggggacccgaggggactcgttaacgaccagccaggccctgcagctggcaggctgcttgagccaactccatagctggtcggtccgggacgtcgaccgtccgacgaactcggttgaggtatcgggccttttcctctaccaggggctcctgcaggccctgaacgggacctccccggaaaaggagatggtccccgaggacgtccgggacttgccctggaggcccgagttgggtcccaccttggacacactgcagctggacgtcgccgacgggctcaacccagggtggaacctgtgtgacgtcgacctgcagcggctgtttgccaccaccatctggcagcagatggaagaactgggaatggcccctaaacggtggtggtagaccgtcgtctaccttcttgacccttaccggggagccctgcagcccacccagggtgccatgccggccttcgcctctgctttccgggacgtcgggtgggtcccacggtacggccggaagcggagacgaaagcagcgccgggcaggaggggtcctggttgcctcccatctgcagagcttcgtcgcggcccgtcctccccaggaccaacggagggtagacgtctcgaagctggaggtgtcgtaccgcgtcttaaggcaccttgcccagcccgacctccacagcatggcgcagaattccgtggaacgggtcggg

SEQ ID NO: 17 - DNA, 1762 bp, Artificial Sequence
  synthetic construct
gatgcgcacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataatgac

SEQ ID NO: 18 - protein, 232 aa, Artificial Sequence
  synthetic construct
AEPKSCDKTHTCPPCPAPEKGGPSVFLFPPKPKDTKMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 229 aa, Artificial Sequence
  synthetic contruct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 585 aa, Artificial Sequence
  synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFENLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 21 - DNA, 703 bp, Artificial Sequence
  synthetic construct
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgatagt

SEQ ID NO: 22 - DNA, 981 bp, Artificial Sequence
  synthetic construct
tccaccaagggcccatcggtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 23 - protein, 406 aa, Artificial Sequence
  synthetic construct
TPLGPASSLPQSFLLKALEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 403 aa, Artificial Sequence
  synthetic construct
TPLGPASSLPQSFLLKALEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 500 aa, Artificial Sequence
  synthetic construct
TPLGPASSLPQSFLLKALEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVE

SEQ ID NO: 26 - DNA, 69 bp, Artificial Sequence
  synthetic construct
gtaagcttgcgtcgacgctagcggcgcgccgccatggccggacctgccacccagagccccatgaagctg

SEQ ID NO: 27 - DNA, 61 bp, Artificial Sequence
  synthetic construct
ggggcagggagctggctgggcccagtggagtggcttcctgcactgtccagagtgcactgtg

SEQ ID NO: 28 - DNA, 59 bp, Artificial Sequence
  synthetic construct
ggacagtgcaggaagccactccactgggcccagccagctccctgccccagagcttcctg

SEQ ID NO: 29 - DNA, 72 bp, Artificial Sequence
  synthetic construct
gaacctcgaggatcctcattagggctgggcaaggtgccttaagacgcggtacgacacctccaggaagctctg

SEQ ID NO: 30 - DNA, 69 bp, Artificial Sequence; identical to SEQ ID NO: 26
  synthetic construct

SEQ ID NO: 31 - DNA, 57 bp, Artificial Sequence
  synthetic construct
gctctaaggccttgagcaggaagctctggggcagggagctcgctgggcccagtggag

SEQ ID NO: 32 - DNA, 53 bp, Artificial Sequence
  synthetic construct
gggcccagcgagctccctgccccagagcttcctgctcaaggccttagagcaag

SEQ ID NO: 33 - DNA, 72 bp, Artificial Sequence; identical to SEQ ID NO: 29
  synthetic construct

SEQ ID NO: 34 - DNA, 69 bp, Artificial Sequence; identical to SEQ ID NO: 26
  synthetic construct

SEQ ID NO: 35 - DNA, 61 bp, Artificial Sequence
  synthetic construct
gtccgagcagcactagttcctcggggtggcacagcttggtggtgttacacagcttctcctg

SEQ ID NO: 36 - DNA, 66 bp, Artificial Sequence
  synthetic construct
ggcgcagcgctccaggagaagctgtgtaacaccaccaagctgtgccaccccgaggaactagtgctg

SEQ ID NO: 37 - DNA, 72 bp, Artificial Sequence; identical to SEQ ID NO: 29
  synthetic construct

SEQ ID NO: 38 - DNA, 69 bp, Artificial Sequence; identical to SEQ ID NO: 26
  synthetic construct

SEQ ID NO: 39 - DNA, 61 bp, Artificial Sequence
  synthetic construct
gcccggcgctggaaagcgctggcgaaggccggcatggcggtctggttgggctgcagggcag

SEQ ID NO: 40 - DNA, 60 bp, Artificial Sequence
  synthetic construct
ggcccctgccctgcagcccaaccagaccgccatgccggccttcgccagcgctttccagcg

SEQ ID NO: 41 - DNA, 72 bp, Artificial Sequence; identical to SEQ ID NO: 29
  synthetic construct

SEQ ID NO: 42 - DNA, 69 bp, Artificial Sequence; identical to SEQ ID NO: 26
  synthetic construct

SEQ ID NO: 43 - DNA, 68 bp, Artificial Sequence
  synthetic construct
gcccggcgctggaaggtagagttgaaggccggcatggcaccctgggtgggctgaagagcaggggccat

SEQ ID NO: 44 - DNA, 74 bp, Artificial Sequence
  synthetic construct
gggaatggcccctgctcttcagcccacccagggtgccatgccggccttcaactctaccttccagcgccgggcag

SEQ ID NO: 45 - DNA, 72 bp, Artificial Sequence; identical to SEQ ID NO: 29
  synthetic construct

SEQ ID NO: 46 - DNA, 20 bp, Artificial Sequence
  synthetic construct
gctagcggcgcgccaccatg

SEQ ID NO: 47 - DNA, 33 bp, Artificial Sequence
  synthetic construct
gctcagggtagcgttaacgatgcccagagagtg

SEQ ID NO: 48 - DNA, 30 bp, Artificial Sequence
  synthetic construct
gggcatcgttaacgctaccctgagcagctg

SEQ ID NO: 49 - DNA, 27 bp, Artificial Sequence
  synthetic construct
gactcgaggatcctcattagggctggg

SEQ ID NO: 50 - DNA, 38 bp, Artificial Sequence
  synthetic construct
gctagcggcgcgccaccatggccggacctgccacccag

SEQ ID NO: 51 - DNA, 45 bp, Artificial Sequence
  synthetic construct
caagcagccggccagctgggtggcgttgctggggcagctgctcag

SEQ ID NO: 52 - DNA, 37 bp, Artificial Sequence
  synthetic construct
gccccagcaacgccacccagctggccggctgcttgag

SEQ ID NO: 53 - DNA, 47 bp, Artificial Sequence
  synthetic construct
gactcgaggatcctcattagggctgggcaaggtgccttaagacgcgg

SEQ ID NO: 54 - DNA, 20 bp, Artificial Sequence
  synthetic construct
gctagcggcgcgccaccatg

SEQ ID NO: 55 - DNA, 27 bp, Artificial Sequence
  synthetic construct
ggggcaactagtcaggttagcccaggg

SEQ ID NO: 56 - DNA, 27 bp, Artificial Sequence
  synthetic construct
gctaacctgactagttgccccagccag

SEQ ID NO: 57 - DNA, 27 bp, Artificial Sequence
  synthetic construct
gactcgaggatcctcattagggctggg

SEQ ID NO: 58 - DNA, 20 bp, Artificial Sequence
  synthetic construct
gctagcggcgcgccaccatg

SEQ ID NO: 59 - DNA, 25 bp, Artificial Sequence
  synthetic construct
ggtgcaattgctcaggggagcccag

SEQ ID NO: 60 - DNA, 23 bp, Artificial Sequence
  synthetic construct
gcaattgcaccagccaggccctg

SEQ ID NO: 61 - DNA, 27 bp, Artificial Sequence
  synthetic construct
gactcgaggatcctcattagggctggg

SEQ ID NO: 62 - DNA, 38 bp, Artificial Sequence; identical to SEQ ID NO: 50
  synthetic construct

SEQ ID NO: 63 - DNA, 37 bp, Artificial Sequence
  synthetic construct
ccggactggtcccgttcagggcctgcaggagcccctg

SEQ ID NO: 64 - DNA, 35 bp, Artificial Sequence
  synthetic construct
gaacgggaccagtccggagttgggtcccaccttgg

SEQ ID NO: 65 - DNA, 47 bp, Artificial Sequence; identical to SEQ ID NO: 53
  synthetic construct

SEQ ID NO: 66 - DNA, 36 bp, Artificial Sequence
  synthetic construct
gtcgacgctagcggcgcgccaccatggccggacctg